SEQUENCE LISTING (19 sequences)

SEQ ID NO: 1 - DNA, 1209 bp, Artificial Sequence
  Synthetic - dGZMB-GSL-MS2CP(N55K)
atgcaaccaatcctgcttctgctggccttcctcctgctgcccagggcagatgcaggggagatcatcgggggacatgaggccaagccccactcccgcccctacatggcttatcttatgatctgggatcagaagtctctgaagaggtgcggtggcttcctgatacgagacgacttcgtgctgacagctgctcactgttggggaagctccataaatgtcaccttgggggcccacaatatcaaagaacaggagccgacccagcagtttatccctgtgaaaagacccatcccccatccagcctataatcctaagaacttctccaacgacatcatgctactgcagctggagagaaaggccaagcggaccagagctgtgcagcccctcaggctacctagcaacaaggcccaggtgaagccagggcagacatgcagtgtggccggctgggggcagacggcccccctgggaaaacactcacacacactacaagaggtgaagatgacagtgcaggaagatcgaaagtgcgaatctgacttacgccattattacgacagtaccattgagttgtgcgtgggggacccagagattaaaaagacttcctttaagggggacgctggaggccctcttgtgtgtaacaaggtggcccagggcattgtctcctatggacgaaacaatggcatgcctccacgagcctgcaccaaagtctcaagctttgtacactggataaagaaaaccatgaaacgctacgcggccgctggaggtgggggttctggcgggggtggatcagggggtggaggttccggtggaggtgggtcgggcgcgcccgcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctactaa

SEQ ID NO: 2 - DNA, 1539 bp, Artificial Sequence
  Synthetic - dGZMB-GSL-eGFP
atgcaaccaatcctgcttctgctggccttcctcctgctgcccagggcagatgcaggggagatcatcgggggacatgaggccaagccccactcccgcccctacatggcttatcttatgatctgggatcagaagtctctgaagaggtgcggtggcttcctgatacgagacgacttcgtgctgacagctgctcactgttggggaagctccataaatgtcaccttgggggcccacaatatcaaagaacaggagccgacccagcagtttatccctgtgaaaagacccatcccccatccagcctataatcctaagaacttctccaacgacatcatgctactgcagctggagagaaaggccaagcggaccagagctgtgcagcccctcaggctacctagcaacaaggcccaggtgaagccagggcagacatgcagtgtggccggctgggggcagacggcccccctgggaaaacactcacacacactacaagaggtgaagatgacagtgcaggaagatcgaaagtgcgaatctgacttacgccattattacgacagtaccattgagttgtgcgtgggggacccagagattaaaaagacttcctttaagggggacgctggaggccctcttgtgtgtaacaaggtggcccagggcattgtctcctatggacgaaacaatggcatgcctccacgagcctgcaccaaagtctcaagctttgtacactggataaagaaaaccatgaaacgctacgcggccgctggaggtgggggttctggcgggggtggatcagggggtggaggttccggtggaggtgggtcgggcgcgcccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 3 - DNA, 5064 bp, Artificial Sequence
  Synthetic -dGZMB-GSL-Cas9
atgcaaccaatcctgcttctgctggccttcctcctgctgcccagggcagatgcaggggagatcatcgggggacatgaggccaagccccactcccgcccctacatggcttatcttatgatctgggatcagaagtctctgaagaggtgcggtggcttcctgatacgagacgacttcgtgctgacagctgctcactgttggggaagctccataaatgtcaccttgggggcccacaatatcaaagaacaggagccgacccagcagtttatccctgtgaaaagacccatcccccatccagcctataatcctaagaacttctccaacgacatcatgctactgcagctggagagaaaggccaagcggaccagagctgtgcagcccctcaggctacctagcaacaaggcccaggtgaagccagggcagacatgcagtgtggccggctgggggcagacggcccccctgggaaaacactcacacacactacaagaggtgaagatgacagtgcaggaagatcgaaagtgcgaatctgacttacgccattattacgacagtaccattgagttgtgcgtgggggacccagagattaaaaagacttcctttaagggggacgctggaggccctcttgtgtgtaacaaggtggcccagggcattgtctcctatggacgaaacaatggcatgcctccacgagcctgcaccaaagtctcaagctttgtacactggataaagaaaaccatgaaacgctacgcggccgctggaggtgggggttctggcgggggtggatcagggggtggaggttccggtggaggtgggtcgggcgcgcccatggcccccaagaagaagcggaaggtgggcatccacggcgtgcccgccgccgacaagaagtacagcatcggcctggacatcggcaccaacagcgtgggctgggccgtgatcaccgacgagtacaaggtgcccagcaagaagttcaaggtgctgggcaacaccgaccggcacagcatcaagaagaacctgatcggcgccctgctgttcgacagcggcgagaccgccgaggccacccggctgaagcggaccgcccggcggcggtacacccggcggaagaaccggatctgctacctgcaggagatcttcagcaacgagatggccaaggtggacgacagcttcttccaccggctggaggagagcttcctggtggaggaggacaagaagcacgagcggcaccccatcttcggcaacatcgtggacgaggtggcctaccacgagaagtaccccaccatctaccacctgcggaagaagctggtggacagcaccgacaaggccgacctgcggctgatctacctggccctggcccacatgatcaagttccggggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccggctgagcaagagccggcggctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgggtgaacaccgagatcaccaaggcccccctgagcgccagcatgatcaagcggtacgacgagcaccaccaggacctgaccctgctgaaggccctggtgcggcagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgggaggacctgctgcggaagcagcggaccttcgacaacggcagcatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaacagccggttcgcctggatgacccggaagagcgaggagaccatcaccccctggaacttcgaggaggtggtggacaagggcgccagcgcccagagcttcatcgagcggatgaccaacttcgacaagaacctgcccaacgagaaggtgctgcccaagcacagcctgctgtacgagtacttcaccgtgtacaacgagctgaccaaggtgaagtacgtgaccgagggcatgcggaagcccgccttcctgagcggcgagcagaagaaggccatcgtggacctgctgttcaagaccaaccggaaggtgaccgtgaagcagctgaaggaggactacttcaagaagatcgagtgcttcgacagcgtggagatcagcggcgtggaggaccggttcaacgccagcctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgagccggaagctgatcaacggcatccgggacaagcagagcggcaagaccatcctggacttcctgaagagcgacggcttcgccaaccggaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaacagccgggagcggatgaagcggatcgaggagggcatcaaggagctgggcagccagatcctgaaggagcaccccgtggagaacacccagctgcagaacgagaagctgtacctgtactacctgcagaacggccgggacatgtacgtggaccaggagctggacatcaaccggctgagcgactacgacgtggaccacatcgtgccccagagcttcctgaaggacgacagcatcgacaacaaggtgctgacccggagcgacaagaaccggggcaagagcgacaacgtgcccagcgaggaggtggtgaagaagatgaagaactactggcggcagctgctgaacgccaagctgatcacccagcggaagttcgacaacctgaccaaggccgagcggggcggcctgagcgagctggacaaggccggcttcatcaagcggcagctggtggagacccggcagatcaccaagcacgtggcccagatcctggacagccggatgaacaccaagtacgacgagaacgacaagctgatccgggaggtgaaggtgatcaccctgaagagcaagctggtgagcgacttccggaaggacttccagttctacaaggtgcgggagatcaacaactaccaccacgcccacgacgcctacctgaacgccgtggtgggcaccgccctgatcaagaagtaccccaagctggagagcgagttcgtgtacggcgactacaaggtgtacgacgtgcggaagatgatcgccaagagcgagcaggagatcggcaaggccaccgccaagtacttcttctacagcaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgagcatgccccaggtgaacatcgtgaagaagaccgaggtgcagaccggcggcttcagcaaggagagcatcctgcccaagcggaacagcgacaagctgatcgcccggaagaaggactgggaccccaagaagtacggcggcttcgacagccccaccgtggcctacagcgtgctggtggtggccaaggtggagaagggcaagagcaagaagctgaagagcgtgaaggagctgctgggcatcaccatcatggagcggagcagcttcgagaagaaccccatcgacttcctggaggccaagggctacaaggaggtgaagaaggacctgatcatcaagctgcccaagtacagcctgttcgagctggagaacggccggaagcggatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgggtgatcctggccgacgccaacctggacaaggtgctgagcgcctacaacaagcaccgggacaagcccatccgggagcaggccgagaacatcatccacctgttcaccctgaccaacctgggcgcccccgccgccttcaagtacttcgacaccaccatcgaccggaagcggtacaccagcaccaaggaggtgctggacgccaccctgatccaccagagcatcaccggcctgtacgagacccggatcgacctgagccagctgggcggcgacagcggcggcaagcggcccgccgccaccaagaaggccggccaggccaagaagaagaagggcagctacccctacgacgtgcccgactacgcctga

SEQ ID NO: 4 - DNA, 1044 bp, Artificial Sequence
  Synthetic -EGFP-MS2SLx6
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatacggtacttattgccaagaaagcacgagcatcagccgtgcctccaggtcgaatcttcaaacgacgacgatcacgcgtcgctccagtattccagggttcatcagatcctacggtacttattgccaagaaagcacgagcatcagccgtgcctccaggtcgaatcttcaaacgacgacgatcacgcgtcgctccagtattccagggttcatcagatcctacggtacttattgccaagaaagcacgagcatcagccgtgcctccaggtcgaatcttcaaacgacgacgatcacgcgtcgctccagtattccagggttcatcagatcc

SEQ ID NO: 5 - DNA, 1368 bp, Artificial Sequence
  Synthetic - EGFP-MS2SLx12
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatacggtacttattgccaagaaagcacgagcatcagccgtgcctccaggtcgaatcttcaaacgacgacgatcacgcgtcgctccagtattccagggttcatcagatcctacggtacttattgccaagaaagcacgagcatcagccgtgcctccaggtcgaatcttcaaacgacgacgatcacgcgtcgctccagtattccagggttcatcagatcctacggtacttattgccaagaaagcacgagcatcagccgtgcctccaggtcgaatcttcaaacgacgacgatcacgcgtcgctccagtattccagggttcatcagatcctacggtacttattgccaagaaagcacgagcatcagccgtgcctccaggtcgaatcttcaaacgacgacgatcacgcgtcgctccagtattccagggttcatcagatcctacggtacttattgccaagaaagcacgagcatcagccgtgcctccaggtcgaatcttcaaacgacgacgatcacgcgtcgctccagtattccagggttcatcagatcctacggtacttattgccaagaaagcacgagcatcagccgtgcctccaggtcgaatcttcaaacgacgacgatcacgcgtcgctccagtattccagggttcatcagatcc

SEQ ID NO: 6 - DNA, 2016 bp, Artificial Sequence
  Synthetic - EGFP-MS2SLx24
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatacggtacttattgccaagaaagcacgagcatcagccgtgcctccaggtcgaatcttcaaacgacgacgatcacgcgtcgctccagtattccagggttcatcagatcctacggtacttattgccaagaaagcacgagcatcagccgtgcctccaggtcgaatcttcaaacgacgacgatcacgcgtcgctccagtattccagggttcatcagatcctacggtacttattgccaagaaagcacgagcatcagccgtgcctccaggtcgaatcttcaaacgacgacgatcacgcgtcgctccagtattccagggttcatcagatcctacggtacttattgccaagaaagcacgagcatcagccgtgcctccaggtcgaatcttcaaacgacgacgatcacgcgtcgctccagtattccagggttcatcagatcctacggtacttattgccaagaaagcacgagcatcagccgtgcctccaggtcgaatcttcaaacgacgacgatcacgcgtcgctccagtattccagggttcatcagatcctacggtacttattgccaagaaagcacgagcatcagccgtgcctccaggtcgaatcttcaaacgacgacgatcacgcgtcgctccagtattccagggttcatcagatcctacggtacttattgccaagaaagcacgagcatcagccgtgcctccaggtcgaatcttcaaacgacgacgatcacgcgtcgctccagtattccagggttcatcagatcctacggtacttattgccaagaaagcacgagcatcagccgtgcctccaggtcgaatcttcaaacgacgacgatcacgcgtcgctccagtattccagggttcatcagatcctacggtacttattgccaagaaagcacgagcatcagccgtgcctccaggtcgaatcttcaaacgacgacgatcacgcgtcgctccagtattccagggttcatcagatcctacggtacttattgccaagaaagcacgagcatcagccgtgcctccaggtcgaatcttcaaacgacgacgatcacgcgtcgctccagtattccagggttcatcagatcctacggtacttattgccaagaaagcacgagcatcagccgtgcctccaggtcgaatcttcaaacgacgacgatcacgcgtcgctccagtattccagggttcatcagatcctacggtacttattgccaagaaagcacgagcatcagccgtgcctccaggtcgaatcttcaaacgacgacgatcacgcgtcgctccagtattccagggttcatcagatcc

SEQ ID NO: 7 - DNA, 5541 bp, Artificial Sequence
  Synthetic - Cas9-MS2SLx24
atggcccccaagaagaagcggaaggtgggcatccacggcgtgcccgccgccgacaagaagtacagcatcggcctggacatcggcaccaacagcgtgggctgggccgtgatcaccgacgagtacaaggtgcccagcaagaagttcaaggtgctgggcaacaccgaccggcacagcatcaagaagaacctgatcggcgccctgctgttcgacagcggcgagaccgccgaggccacccggctgaagcggaccgcccggcggcggtacacccggcggaagaaccggatctgctacctgcaggagatcttcagcaacgagatggccaaggtggacgacagcttcttccaccggctggaggagagcttcctggtggaggaggacaagaagcacgagcggcaccccatcttcggcaacatcgtggacgaggtggcctaccacgagaagtaccccaccatctaccacctgcggaagaagctggtggacagcaccgacaaggccgacctgcggctgatctacctggccctggcccacatgatcaagttccggggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccggctgagcaagagccggcggctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgggtgaacaccgagatcaccaaggcccccctgagcgccagcatgatcaagcggtacgacgagcaccaccaggacctgaccctgctgaaggccctggtgcggcagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgggaggacctgctgcggaagcagcggaccttcgacaacggcagcatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaacagccggttcgcctggatgacccggaagagcgaggagaccatcaccccctggaacttcgaggaggtggtggacaagggcgccagcgcccagagcttcatcgagcggatgaccaacttcgacaagaacctgcccaacgagaaggtgctgcccaagcacagcctgctgtacgagtacttcaccgtgtacaacgagctgaccaaggtgaagtacgtgaccgagggcatgcggaagcccgccttcctgagcggcgagcagaagaaggccatcgtggacctgctgttcaagaccaaccggaaggtgaccgtgaagcagctgaaggaggactacttcaagaagatcgagtgcttcgacagcgtggagatcagcggcgtggaggaccggttcaacgccagcctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgagccggaagctgatcaacggcatccgggacaagcagagcggcaagaccatcctggacttcctgaagagcgacggcttcgccaaccggaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaacagccgggagcggatgaagcggatcgaggagggcatcaaggagctgggcagccagatcctgaaggagcaccccgtggagaacacccagctgcagaacgagaagctgtacctgtactacctgcagaacggccgggacatgtacgtggaccaggagctggacatcaaccggctgagcgactacgacgtggaccacatcgtgccccagagcttcctgaaggacgacagcatcgacaacaaggtgctgacccggagcgacaagaaccggggcaagagcgacaacgtgcccagcgaggaggtggtgaagaagatgaagaactactggcggcagctgctgaacgccaagctgatcacccagcggaagttcgacaacctgaccaaggccgagcggggcggcctgagcgagctggacaaggccggcttcatcaagcggcagctggtggagacccggcagatcaccaagcacgtggcccagatcctggacagccggatgaacaccaagtacgacgagaacgacaagctgatccgggaggtgaaggtgatcaccctgaagagcaagctggtgagcgacttccggaaggacttccagttctacaaggtgcgggagatcaacaactaccaccacgcccacgacgcctacctgaacgccgtggtgggcaccgccctgatcaagaagtaccccaagctggagagcgagttcgtgtacggcgactacaaggtgtacgacgtgcggaagatgatcgccaagagcgagcaggagatcggcaaggccaccgccaagtacttcttctacagcaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgagcatgccccaggtgaacatcgtgaagaagaccgaggtgcagaccggcggcttcagcaaggagagcatcctgcccaagcggaacagcgacaagctgatcgcccggaagaaggactgggaccccaagaagtacggcggcttcgacagccccaccgtggcctacagcgtgctggtggtggccaaggtggagaagggcaagagcaagaagctgaagagcgtgaaggagctgctgggcatcaccatcatggagcggagcagcttcgagaagaaccccatcgacttcctggaggccaagggctacaaggaggtgaagaaggacctgatcatcaagctgcccaagtacagcctgttcgagctggagaacggccggaagcggatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgggtgatcctggccgacgccaacctggacaaggtgctgagcgcctacaacaagcaccgggacaagcccatccgggagcaggccgagaacatcatccacctgttcaccctgaccaacctgggcgcccccgccgccttcaagtacttcgacaccaccatcgaccggaagcggtacaccagcaccaaggaggtgctggacgccaccctgatccaccagagcatcaccggcctgtacgagacccggatcgacctgagccagctgggcggcgacagcggcggcaagcggcccgccgccaccaagaaggccggccaggccaagaagaagaagggcagctacccctacgacgtgcccgactacgcctgatacggtacttattgccaagaaagcacgagcatcagccgtgcctccaggtcgaatcttcaaacgacgacgatcacgcgtcgctccagtattccagggttcatcagatcctacggtacttattgccaagaaagcacgagcatcagccgtgcctccaggtcgaatcttcaaacgacgacgatcacgcgtcgctccagtattccagggttcatcagatcctacggtacttattgccaagaaagcacgagcatcagccgtgcctccaggtcgaatcttcaaacgacgacgatcacgcgtcgctccagtattccagggttcatcagatcctacggtacttattgccaagaaagcacgagcatcagccgtgcctccaggtcgaatcttcaaacgacgacgatcacgcgtcgctccagtattccagggttcatcagatcctacggtacttattgccaagaaagcacgagcatcagccgtgcctccaggtcgaatcttcaaacgacgacgatcacgcgtcgctccagtattccagggttcatcagatcctacggtacttattgccaagaaagcacgagcatcagccgtgcctccaggtcgaatcttcaaacgacgacgatcacgcgtcgctccagtattccagggttcatcagatcctacggtacttattgccaagaaagcacgagcatcagccgtgcctccaggtcgaatcttcaaacgacgacgatcacgcgtcgctccagtattccagggttcatcagatcctacggtacttattgccaagaaagcacgagcatcagccgtgcctccaggtcgaatcttcaaacgacgacgatcacgcgtcgctccagtattccagggttcatcagatcctacggtacttattgccaagaaagcacgagcatcagccgtgcctccaggtcgaatcttcaaacgacgacgatcacgcgtcgctccagtattccagggttcatcagatcctacggtacttattgccaagaaagcacgagcatcagccgtgcctccaggtcgaatcttcaaacgacgacgatcacgcgtcgctccagtattccagggttcatcagatcctacggtacttattgccaagaaagcacgagcatcagccgtgcctccaggtcgaatcttcaaacgacgacgatcacgcgtcgctccagtattccagggttcatcagatcctacggtacttattgccaagaaagcacgagcatcagccgtgcctccaggtcgaatcttcaaacgacgacgatcacgcgtcgctccagtattccagggttcatcagatcc

SEQ ID NO: 8 - DNA, 428 bp, Artificial Sequence
  Synthetic - U6-B2MsgRNA-MS2SL
aaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgactcacgctggatagcctccgttttagagctaggccaacatgaggatcacccatgtctgcagggcctagcaagttaaaataaggctagtccgttatcaacttggccaacatgaggatcacccatgtctgcagggccaagtggcaccgagtcggtgcttttttt

SEQ ID NO: 9 - DNA, 20 bp, Artificial Sequence
  Synthetic - single guide RNA for knock down of GZMA
accatgtagggtcttgaatg

SEQ ID NO: 10 - DNA, 20 bp, Artificial Sequence
  Synthetic - single guide RNA for knock down of GZMB
tttccttcaggggagatcat

SEQ ID NO: 11 - DNA, 20 bp, Artificial Sequence
  Synthetic - single guide RNA for knock down of GZMM
ccccagcaccagcctcagct

SEQ ID NO: 12 - DNA, 741 bp, Artificial Sequence
  Synthetic - catalytically inactive granzyme B (dGZMB)
atgcaaccaatcctgcttctgctggccttcctcctgctgcccagggcagatgcaggggagatcatcgggggacatgaggccaagccccactcccgcccctacatggcttatcttatgatctgggatcagaagtctctgaagaggtgcggtggcttcctgatacgagacgacttcgtgctgacagctgctcactgttggggaagctccataaatgtcaccttgggggcccacaatatcaaagaacaggagccgacccagcagtttatccctgtgaaaagacccatcccccatccagcctataatcctaagaacttctccaacgacatcatgctactgcagctggagagaaaggccaagcggaccagagctgtgcagcccctcaggctacctagcaacaaggcccaggtgaagccagggcagacatgcagtgtggccggctgggggcagacggcccccctgggaaaacactcacacacactacaagaggtgaagatgacagtgcaggaagatcgaaagtgcgaatctgacttacgccattattacgacagtaccattgagttgtgcgtgggggacccagagattaaaaagacttcctttaagggggacgctggaggccctcttgtgtgtaacaaggtggcccagggcattgtctcctatggacgaaacaatggcatgcctccacgagcctgcaccaaagtctcaagctttgtacactggataaagaaaaccatgaaacgctac

SEQ ID NO: 13 - DNA, 78 bp, Artificial Sequence
  Synthetic - flexible glycine serine linker (GSL)
gcggccgctggaggtgggggttctggcgggggtggatcagggggtggaggttccggtggaggtgggtcgggcgcgccc

SEQ ID NO: 14 - DNA, 387 bp, Artificial Sequence
  Synthetic - MS2 coat protein (MS2CP) containing high affinity N55K mutation
gcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctac

SEQ ID NO: 15 - DNA, 720 bp, Artificial Sequence
  Synthetic - eGFP
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 16 - DNA, 4245 bp, Artificial Sequence
  Synthetic - Cas9
atggcccccaagaagaagcggaaggtgggcatccacggcgtgcccgccgccgacaagaagtacagcatcggcctggacatcggcaccaacagcgtgggctgggccgtgatcaccgacgagtacaaggtgcccagcaagaagttcaaggtgctgggcaacaccgaccggcacagcatcaagaagaacctgatcggcgccctgctgttcgacagcggcgagaccgccgaggccacccggctgaagcggaccgcccggcggcggtacacccggcggaagaaccggatctgctacctgcaggagatcttcagcaacgagatggccaaggtggacgacagcttcttccaccggctggaggagagcttcctggtggaggaggacaagaagcacgagcggcaccccatcttcggcaacatcgtggacgaggtggcctaccacgagaagtaccccaccatctaccacctgcggaagaagctggtggacagcaccgacaaggccgacctgcggctgatctacctggccctggcccacatgatcaagttccggggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccggctgagcaagagccggcggctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgggtgaacaccgagatcaccaaggcccccctgagcgccagcatgatcaagcggtacgacgagcaccaccaggacctgaccctgctgaaggccctggtgcggcagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgggaggacctgctgcggaagcagcggaccttcgacaacggcagcatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaacagccggttcgcctggatgacccggaagagcgaggagaccatcaccccctggaacttcgaggaggtggtggacaagggcgccagcgcccagagcttcatcgagcggatgaccaacttcgacaagaacctgcccaacgagaaggtgctgcccaagcacagcctgctgtacgagtacttcaccgtgtacaacgagctgaccaaggtgaagtacgtgaccgagggcatgcggaagcccgccttcctgagcggcgagcagaagaaggccatcgtggacctgctgttcaagaccaaccggaaggtgaccgtgaagcagctgaaggaggactacttcaagaagatcgagtgcttcgacagcgtggagatcagcggcgtggaggaccggttcaacgccagcctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgagccggaagctgatcaacggcatccgggacaagcagagcggcaagaccatcctggacttcctgaagagcgacggcttcgccaaccggaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaacagccgggagcggatgaagcggatcgaggagggcatcaaggagctgggcagccagatcctgaaggagcaccccgtggagaacacccagctgcagaacgagaagctgtacctgtactacctgcagaacggccgggacatgtacgtggaccaggagctggacatcaaccggctgagcgactacgacgtggaccacatcgtgccccagagcttcctgaaggacgacagcatcgacaacaaggtgctgacccggagcgacaagaaccggggcaagagcgacaacgtgcccagcgaggaggtggtgaagaagatgaagaactactggcggcagctgctgaacgccaagctgatcacccagcggaagttcgacaacctgaccaaggccgagcggggcggcctgagcgagctggacaaggccggcttcatcaagcggcagctggtggagacccggcagatcaccaagcacgtggcccagatcctggacagccggatgaacaccaagtacgacgagaacgacaagctgatccgggaggtgaaggtgatcaccctgaagagcaagctggtgagcgacttccggaaggacttccagttctacaaggtgcgggagatcaacaactaccaccacgcccacgacgcctacctgaacgccgtggtgggcaccgccctgatcaagaagtaccccaagctggagagcgagttcgtgtacggcgactacaaggtgtacgacgtgcggaagatgatcgccaagagcgagcaggagatcggcaaggccaccgccaagtacttcttctacagcaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgagcatgccccaggtgaacatcgtgaagaagaccgaggtgcagaccggcggcttcagcaaggagagcatcctgcccaagcggaacagcgacaagctgatcgcccggaagaaggactgggaccccaagaagtacggcggcttcgacagccccaccgtggcctacagcgtgctggtggtggccaaggtggagaagggcaagagcaagaagctgaagagcgtgaaggagctgctgggcatcaccatcatggagcggagcagcttcgagaagaaccccatcgacttcctggaggccaagggctacaaggaggtgaagaaggacctgatcatcaagctgcccaagtacagcctgttcgagctggagaacggccggaagcggatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgggtgatcctggccgacgccaacctggacaaggtgctgagcgcctacaacaagcaccgggacaagcccatccgggagcaggccgagaacatcatccacctgttcaccctgaccaacctgggcgcccccgccgccttcaagtacttcgacaccaccatcgaccggaagcggtacaccagcaccaaggaggtgctggacgccaccctgatccaccagagcatcaccggcctgtacgagacccggatcgacctgagccagctgggcggcgacagcggcggcaagcggcccgccgccaccaagaaggccggccaggccaagaagaagaagggcagctacccctacgacgtgcccgactacgcctga

SEQ ID NO: 17 - DNA, 324 bp, Artificial Sequence
  Synthetic - MS2SLx6
tacggtacttattgccaagaaagcacgagcatcagccgtgcctccaggtcgaatcttcaaacgacgacgatcacgcgtcgctccagtattccagggttcatcagatcctacggtacttattgccaagaaagcacgagcatcagccgtgcctccaggtcgaatcttcaaacgacgacgatcacgcgtcgctccagtattccagggttcatcagatcctacggtacttattgccaagaaagcacgagcatcagccgtgcctccaggtcgaatcttcaaacgacgacgatcacgcgtcgctccagtattccagggttcatcagatcc

SEQ ID NO: 18 - DNA, 648 bp, Artificial Sequence
  Synthetic - MS2SLx12
tacggtacttattgccaagaaagcacgagcatcagccgtgcctccaggtcgaatcttcaaacgacgacgatcacgcgtcgctccagtattccagggttcatcagatcctacggtacttattgccaagaaagcacgagcatcagccgtgcctccaggtcgaatcttcaaacgacgacgatcacgcgtcgctccagtattccagggttcatcagatcctacggtacttattgccaagaaagcacgagcatcagccgtgcctccaggtcgaatcttcaaacgacgacgatcacgcgtcgctccagtattccagggttcatcagatcctacggtacttattgccaagaaagcacgagcatcagccgtgcctccaggtcgaatcttcaaacgacgacgatcacgcgtcgctccagtattccagggttcatcagatcctacggtacttattgccaagaaagcacgagcatcagccgtgcctccaggtcgaatcttcaaacgacgacgatcacgcgtcgctccagtattccagggttcatcagatcctacggtacttattgccaagaaagcacgagcatcagccgtgcctccaggtcgaatcttcaaacgacgacgatcacgcgtcgctccagtattccagggttcatcagatcc

SEQ ID NO: 19 - DNA, 1296 bp, Artificial Sequence
  Synthetic - MS2SLx24
tacggtacttattgccaagaaagcacgagcatcagccgtgcctccaggtcgaatcttcaaacgacgacgatcacgcgtcgctccagtattccagggttcatcagatcctacggtacttattgccaagaaagcacgagcatcagccgtgcctccaggtcgaatcttcaaacgacgacgatcacgcgtcgctccagtattccagggttcatcagatcctacggtacttattgccaagaaagcacgagcatcagccgtgcctccaggtcgaatcttcaaacgacgacgatcacgcgtcgctccagtattccagggttcatcagatcctacggtacttattgccaagaaagcacgagcatcagccgtgcctccaggtcgaatcttcaaacgacgacgatcacgcgtcgctccagtattccagggttcatcagatcctacggtacttattgccaagaaagcacgagcatcagccgtgcctccaggtcgaatcttcaaacgacgacgatcacgcgtcgctccagtattccagggttcatcagatcctacggtacttattgccaagaaagcacgagcatcagccgtgcctccaggtcgaatcttcaaacgacgacgatcacgcgtcgctccagtattccagggttcatcagatcctacggtacttattgccaagaaagcacgagcatcagccgtgcctccaggtcgaatcttcaaacgacgacgatcacgcgtcgctccagtattccagggttcatcagatcctacggtacttattgccaagaaagcacgagcatcagccgtgcctccaggtcgaatcttcaaacgacgacgatcacgcgtcgctccagtattccagggttcatcagatcctacggtacttattgccaagaaagcacgagcatcagccgtgcctccaggtcgaatcttcaaacgacgacgatcacgcgtcgctccagtattccagggttcatcagatcctacggtacttattgccaagaaagcacgagcatcagccgtgcctccaggtcgaatcttcaaacgacgacgatcacgcgtcgctccagtattccagggttcatcagatcctacggtacttattgccaagaaagcacgagcatcagccgtgcctccaggtcgaatcttcaaacgacgacgatcacgcgtcgctccagtattccagggttcatcagatcctacggtacttattgccaagaaagcacgagcatcagccgtgcctccaggtcgaatcttcaaacgacgacgatcacgcgtcgctccagtattccagggttcatcagatcc

(19) United States
(12) Patent Application Publication
(10) Pub. No.: US 2024/0026292 A1
(43) Pub. Date: Jan. 25, 2024

(54) ENGINEERED IMMUNE CELLS FOR REAGENT DELIVERY

(71) Applicant: REGENTS OF THE UNIVERSITY OF MINNESOTA, Minneapolis, MN (US)

(72) Inventors: Branden MORIARITY, Minneapolis, MN (US); Beau WEBBER, Minneapolis, MN (US); Emily POMEROY, Minneapolis, MN (US)

(73) Assignee: REGENTS OF THE UNIVERSITY OF MINNESOTA, Minneapolis, MN (US)

(21) Appl. No.: 18/044,482

(22) PCT Filed: Sep. 9, 2021
(86) PCT No.: PCT/US2021/049673

§ 371 Date: Mar. 8, 2023

Related U.S. Application Data
(60) Provisional application No. 63/075,905, filed on Sep. 9, 2020.

(51) Int. Cl. C12N 5/078; A61K 39/00; A61K 35/17; C12N 9/22

(52) U.S. Cl. CPC C12N 5/0634; A61K 39/4631; A61K 35/17; C12N 9/22; C12N 2510/00; A61K 2239/15

(57) ABSTRACT

The present invention provides genetically modified lymphocyte with reduced cytotoxicity. Also provided are methods of using these genetically modified lymphocytes to deliver cargo molecules to a target cell.

### CROSS-REFERENCE TO RELATED APPLICATIONS

This application claims priority to U.S. Provisional Application No. 63/075,905 filed on Sep. 9, 2020, the contents of which are incorporated by reference in their entireties.

### STATEMENT REGARDING FEDERALLY SPONSORED RESEARCH

N/A

### SEQUENCE LISTING

A Sequence Listing accompanies this application and is submitted as an ASCII text file of the sequence listing named “920171 00438 ST25.txt” which is 40,960 bytes in size and was created on Sep. 8, 2021. The sequence listing is electronically submitted via EFS-Web with the application and is incorporated herein by reference in its entirety.

### BACKGROUND

The ability to introduce specific sequence changes into a cell's DNA holds great promise for the treatment of human diseases, including conditions driven by known genetic mutations as well as more complex diseases. However, this approach is contingent on efficient delivery of gene editing reagents to target cells and tissues. This can be accomplished either by direct in vivo delivery of constructs encoding gene editing reagents (i.e., nucleases, base editors, prime editors, therapeutic transgenes) or by ex vivo modification of cells and subsequent transplant.

While ex vivo modification is a promising strategy for certain target cell types, it is not an option for many target cells and tissues. Current approaches for in vivo delivery largely rely upon the use of engineered polymers or viral vectors, namely recombinant adeno-associated viruses (rAAV) and lentiviruses. rAAV have been used to target tissues including liver, nervous system, muscle, and retina. Despite some success, these current approaches have several limitations, including those related to immunogenicity, vector carrying capacity, and delivery efficiency. Importantly, it is difficult to be selective about what target cells receive molecules delivered by viruses or polymers.

Accordingly, there remains a need in the art for improved methods for delivering gene editing reagents and therapeutic cargo to target cells in vivo.

### SUMMARY

The present invention provides domesticated genetically modified lymphocytes that are deficient in one or more cytotoxic proteins (e.g., have reduced or eliminated expression) and are capable of delivery of cargo to target cells. These genetically modified lymphocytes can be used to deliver different cargos to different cells, and can be targeted by including a targeting molecule, including, for example, chimeric antigen receptors. Methods of using such genetically modified lymphocytes are also described.

In a first aspect, the present invention provides genetically modified lymphocytes comprising (a) a mutation in one or more cytotoxic proteins that reduces or eliminates the expression of the one or more cytotoxic protein in the lymphocyte; and (b) a cargo molecule. Importantly, the lymphocytes have reduced or no expression of the one or more cytotoxic protein. In some aspects, the cargo molecule is encoded within a construct and operable linked to a promoter, wherein the lymphocyte is capable of expressing the cargo molecule.

In a second aspect, the present invention provides methods for delivering a cargo molecule to a target cell. The methods comprise co-culturing the target cell with a genetically modified lymphocyte described herein, such that the cargo molecule is delivered to the target cell.

In a third aspect, the present invention provides methods for genetically modifying a target cell. The methods comprise contacting the target cell with a genetically modified lymphocyte described herein in which the cargo molecule is a Cas9 protein or base editor, an RNA encoding Cas9, a guide nucleic acid (gNA), or a combination thereof.

In a fourth aspect, the present invention provides methods for delivering one or more gene editing reagent to a target cell. The methods comprise (a) providing a genetically modified lymphocyte comprising one or more gene editing reagents, for example, one or more exogenous nucleic acids encoding (i) a cargo molecule comprising a fusion protein comprising a catalytically dead granzyme (dGZ) fused to gene editing reagent, preferably a Cas9 protein, and one or more gNAs having complementarity to a target nucleic acid sequence to be genetically modified in the target cell; or (ii) a cargo molecule comprising a fusion protein comprising a dGZ and an MS2 RNA-binding protein, and an RNA comprising one or more MS2 stem loops, and/or (iii) a targeting molecule, preferably a chimeric antigen receptor (CAR), wherein the targeting molecule comprises a ligand-binding domain having specificity for an antigen present on the target cell. The genetically modified lymphocyte expresses at last one cargo molecule, a targeting molecule, or both; and (b) incubating the genetically modified lymphocyte with the target cell under conditions that promote activation of the genetically modified lymphocyte and delivery of the one or more cargo molecules (e.g., gene editing reagents) to the target cell, preferably through perforin-induced membrane pores.

In a fifth aspect, the present invention provides genetically modified lymphocytes capable of gene editing a target cell wherein the genetically modified lymphocyte is deficient in one or more cytotoxic proteins. The lymphocytes comprise a cargo molecule, preferably a gene editing reagent. In some aspects, the lymphocyte comprises one or more nucleic acids encoding a (i) the gene editing agent, for example, a fusion protein comprising a catalytically dead granzyme B (dGZMB), granulysin, or catalytically inactive granulysin fused to a gene editing reagent, preferably a Cas9 protein, or dGZMB fused to a MS2 RNA-binding protein, and (ii) one or more gNAs having complementarity to a target nucleic acid sequence to be genetically modified in the target cell. Further, the human lymphocyte can be genetically modified to express a chimeric antigen receptor (CAR) comprising a single-chain variant fragment (scFv) specific for the target cell.

Further aspects are described and detailed herein.

### BRIEF DESCRIPTION OF THE DRAWINGS

FIG. 1 is a schematic depiction of the six constructs used in the Examples. For a detailed description of these constructs, see the section titled Materials and Methods.

FIG. 2 is a graph showing the percent of live T cells expressing the cytotoxic proteins granzyme A (GZMA), granzyme B (GZMB), granzyme K (GZMK), granzyme M (GZMM), Fas ligand (FasL), granulysin (GNLY), perforin, and tumor necrosis factor-related apoptosis-inducing ligand (TRAIL) following stimulation with Dynabeads. Protein expression was quantified on days 0, 1, 3, and 7 using flow cytometry.

FIG. 3 is a graph showing the percent of live CD3+T cells expressing granzymes A, B, and M following Cas9-based gene editing, which was used to knock out these three proteins concurrently in T cells from two donors. These results were generated using flow cytometry. Data are represented as mean±SD, n=3 per donor n=3 control.

FIG. 4 is a graph showing the percent of live CD3+ T cells expressing an anti-CD19 CAR (detected using a transcriptionally linked RQR8 sequence tag) following lentiviral-based introduction of a construct encoding the CAR in the presence or absence of a chaperone construct encoding a catalytically inactive form of granzyme B (dGZMB) linked to GFP (GZMB-GFP) or a positive control construct encoding GFP. These results were generated using flow cytometry. Data are represented as mean±SD, n=6 (3 replicates of 2 donors) per group.

FIG. 5 is a line graph showing the number of apoptotic cells over time in a killing assay. Counts are of Cytolight Rapid Orange labeled CD19+ Raji cells co-labeled with Annexin V dye following co-culture with either (1) GZMA/GZMB/GZMM triple knockout anti-CD19 CAR T cells that express dGZMB-GSL-GFP, (2) wild-type anti-CD19 CART cells, (3) GZMA/GZMB/GZMM triple knockout T cells, or (4) WT T cells. Counts were acquired from multifluorescent images taken every 30 minutes for 48 hours. Significant main effects of group, time, and a group by time interaction are observed. Data represented as mean±SD, n=3 per group per time point.

FIG. 6 is a graph the intracellular GFP signal within target Raji cells that were cultured alone (Raji only) or co-cultured with GZMA/GZMB/GZMM triple knockout T cells (i.e., wild-type or expressing MND-GFP or MND-dGZMB-GFP). Internalized GFP signal in target cells indicates that cargo (i.e., GFP protein) has been transferred across the immunological synapse.

FIG. 7 illustrates use of engineered NK cells to deliver gene editing RNA via the perforin-granzyme pathway. NK cells are engineered to express granzyme B (GZB) fused to a molecule such as a gene editing enzyme or MS2 binding protein, which are packaged and stored in cytolytic granules. Target cells expressing CD34 activate the NK cell via a CD34-specific chimeric antigen receptor (CD34-CAR), and CD34-CAR ligation initiates formation of an immunological synapse and induces release of the cytolytic granules to the target cell. The cargo molecules diffuse into the target cell via the perforin pore.

### DETAILED DESCRIPTION

All publications, including but not limited to patents and patent applications, cited in this specification are herein incorporated by reference as though set forth in their entirety in the present application.

The present invention provides genetically modified lymphocyte with reduced cytotoxicity and methods of using these genetically modified lymphocytes to deliver cargo molecules to a target cell, including, for example, gene editing reagents. The genetically modified lymphocytes described herein are considered “domesticated” as they have been reduced in their ability to target cell, and, as such, can be used as a delivery cell to deliver cargo or choice to a target cell. In other words, the genetically modified lymphocytes have been reducing in their ability to kill target cells, but the mechanism by which they target cells is still functional to allow the lymphocytes to carry and release cargo to target cells.

The methods and compositions described herein are based at least in part on the inventors' development of highly-engineered lymphocytes (“domesticated lymphocytes”) which have reduced cytotoxicity but the reduction or elimination of at least one cytotoxic protein usually expressed in the cytotoxic granules, and methods of using such cells for targeted in vivo delivery of cargo, e.g., proteins, mRNA, DNA and small molecules, including gene editing reagents (i.e., nucleases, base editors, prime editors, therapeutic transgenes) into target immune cells. In some instances, the cargo is provided as constructs encoding the cargo, but other cargo and methods are described and contemplated herein. This allows for these genetically modified lymphocytes to deliver to specific target cells cargo including gene editing reagents allowing for targeted gene editing in specific cells both in vitro and in vivo. Not to be bound by any theory, but the methods and the genetically engineered lymphocytes described herein are believed to be able to package the cargo contained within the lyphocyote by packaging into the lymphocytes cytotoxic granules, which are released when the lymphocyte is able to bind to the target cell and is activated, along with the endogenous perforin allowing the cargo to traverse into the target cell through the perforin-created pores.

Conventional approaches for in vivo gene delivery largely rely upon the use of engineered polymers or viral vectors, namely recombinant adeno-associated viruses (rAAV) and lentiviruses. However, the success of these approaches is limited by immunogenicity, vector carrying capacity, and delivery efficiency. Importantly, it is difficult to be selective about what target cells receive molecules delivered by viruses or polymers, and efficiency can be very low. To overcome these limitations, the inventors have employed genetically engineered lymphocytes, which can go anywhere in the body and identify specific cell types, to selectively deliver cargo molecules to target cells in vivo. In particular, the methods and compositions of the present invention harness elements of the perforin-granzyme pathway.

The granzyme-perforin pathway is the main pathway used by cytotoxic lymphocytes to induce apoptosis in virus-infected or transformed cells. Its main constituent components are serine proteases referred to as granzymes and the pore forming protein perforin, both of which are stored in membrane-bound secretory lysosomes (referred to herein as cytolytic granules) in the cytosol of cytotoxic lymphocytes. Upon target cell recognition, the cytotoxic lymphocyte forms an immune synapse with the target cell (i.e., an interface between a lymphocyte and a target cell). Surface receptor signaling results in the endocytic release of granzymes and perforin from the cytolytic granules into the synapse between these two cells. Perforin then inserts in the target cell membrane and oligomerizes to form transient pores, through which granzymes diffuse into the target cell. Finally, granzymes cleave caspases and BH3-interacting domain death agonist (BID) to initiate target cell apoptosis. Importantly, the effects of the cytotoxic lymphocyte are largely restricted to the target cell only, as surrounding bystander cells typically do not receive appreciable quantities of granzymes.

The present inventors have commandeered this pathway to allow cytoplasm-to-cytoplasm transfer of cargo molecules carried a lymphocyte to a target cell. To prevent this process from killing the target cell, the inventors mutated several key granzymes within the delivery lymphocyte producing a domesticated or less toxic lymphocyte. The resulting genetically modified lymphocytes have reduced cytotoxicity and can, thus, be used to deliver cargo molecules to specific target cells without resulting in toxicity. In some embodiments, the cargo molecule is carried via a fusion protein with a catalytically inactive granzyme molecule.

### Genetically Modified Lymphocytes

In a first aspect, the present invention provides genetically modified lymphocytes comprising: (a) a mutation in one or more cytotoxic protein, wherein the mutation reduces or eliminates the expression of the one or more cytotoxic protein in the lymphocyte (e.g., the lymphocyte comprises a mutation in one or more nucleic acids within the coding region of the one or more cytotoxic proteins (e.g., granzyme, etc.) which reduces or eliminates the cytotoxic protein expression in the cell); and (b) a cargo molecule. Importantly, the genetically modified lymphocytes have reduced or no expression of the one or more cytotoxic protein and the cargo molecule is able to be packaged into the granules within the lymphocyte, allowing for targeted delivery once the lymphocyte binds to a target cell and is activated. In a preferred embodiment, the cargo molecule may be fused to a catalytically inactive granzyme, as described more herein. In some embodiments, the cargo molecule is encoded in a first construct that is introduced into the genetically modified lymphocytes.

A cell is “genetically modified” or “genetically engineered” if the cell includes a modification to its genome compared to an unmodified control cell or if it comprises an exogenous polynucleotide. Genetic modifications, i.e., “mutations”, include base substitutions, deletions, insertions, and integrations of exogenous DNA into the cells genome. In some cases, the cell has been modified to comprise a non-naturally occurring nucleic acid molecule that has been created or modified by the hand of man (e.g., using recombinant DNA technology) or is derived from such a molecule (e.g., by transcription, translation, etc.). A cell that contains an exogenous, recombinant, synthetic, and/or otherwise modified polynucleotide is considered to be an engineered cell. In one embodiment, the genetically modified cells are engineered to deliver nucleic acids, proteins or small molecules via the perforin-granzyme pathway to a target cell. In particular, provided herein are genetically modified cells that can still identify target cells and open pores in the target cells through perforin but, instead of toxic granzymes being delivered, deliver cargo molecules or deliver inactive granzymes fused to different cargo such as proteins, RNA binding proteins (e.g., MS2), mRNA or gene editing reagents.

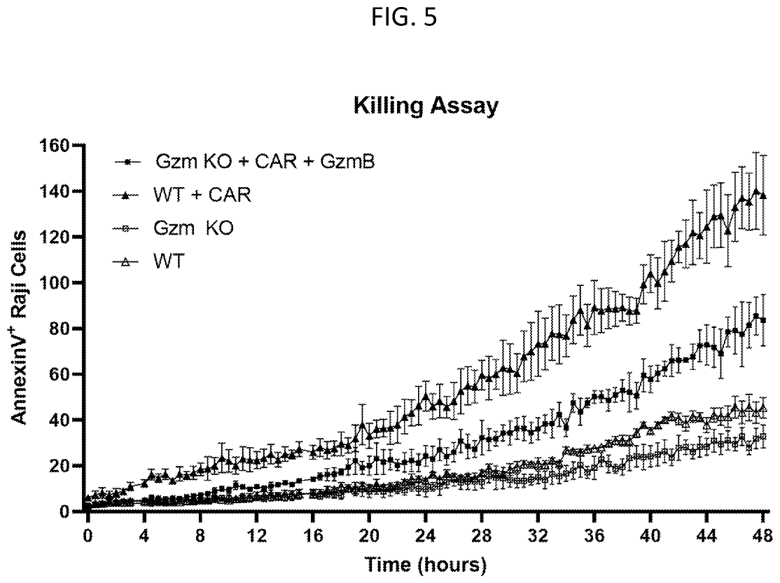

In the examples, the inventors demonstrate that their genetically modified T cells have reduced cytotoxicity (see FIG. 5). Thus, in some embodiments, the genetically modified lymphocytes exhibit reduced cytotoxicity as compared to an unmodified control cell. Reduced cytotoxicity can be measured using a killing assay, i.e., an assay in which living target cells are quantified following co-culture with the lymphocytes to determine whether the genetically modified lymphocytes kill fewer target cells as compared to unmodified control cell. Suitable killing assays include, without limitation, luciferase-based killing assays, imaging-based killing assays, annexin V staining-based killing assays, cleaved caspase staining-based killing assays, chromium release assays, and in vivo toxicity assays.

A “lymphocyte” is type of white blood cell that is of fundamental importance in the immune system. Lymphocytes of the present invention include natural killer cells, T cells, including CD4+ and CD8+ T cells, regulatory T cells, and gamma-delta T cells. In the examples, the inventors generate genetically modified CD4+ and CD8+ T cells that have reduced cytotoxicity. Thus, in some embodiments, the lymphocyte is a T cell.

The lymphocytes used with the present invention may be any source. For example, the lymphocytes may be from a patient or donor or a population of cultured cell. However, to acquire large numbers of highly engineered lymphocytes with ease, it may be advantageous to utilize lymphocytes derived from cultured cells, such as pluripotent stem cells (e.g., embryonic stem cells or induced pluripotent stem cells). Thus, in some embodiments, the lymphocytes were differentiated from pluripotent stem cells. In some embodiments, the lymphocytes were differentiated from induced pluripotent stem cells. Methods of differentiating lymphocytes from pluripotent stem cells are well known in the art. See, e.g., Exp Hematol. (2019) 71:13-23; Curr Hematol Malig Rep. (2019) 14 (4): 261-268; and Nat Commun (2021) 12 (1):430.

As used herein, a “cytotoxic protein” is a protein that damages or kill cells. In some embodiments, the one or more cytotoxic protein that is modified in the lymphocyte is selected from the group consisting of granzyme A (GZMA), granzyme B (GZMB), granzyme K (GZMK), granzyme M (GZMM), or other cytotoxic proteins expressed from a lymphocyte. Granzymes A, B, K, and M are serine proteases that induce caspase cleavage and trigger apoptosis in target cells. The reduction or elimination of the one or more cytotoxic proteins within the lymphocytes results in a functional lymphocyte that is able to dock a target cell but unable to kill or apoptose the target cell by the release of cytotoxic proteins. The resultant lymphocytes thus have reduced or eliminated expression of one or more, two or more, or three or more cytotoxic proteins within the lymphocytes.

In further embodiments, the genetically modified lymphocytes may be further altered to reduce the expression of additional immune regulatory proteins, including, for example, Fas ligand (FasL), granulysin (GNLY), perforin, IFNG, TNFA, tumor necrosis factor-related apoptosis-inducing ligand (TRAIL), IFNγ, TNFα, and tryptase-2, tryptase beta 2 (TPSB2), among others. FasL is a ligand that is expressed on activated T cells and induces apoptosis of cells displaying the Fas receptor. GNLY is expressed by T cells during antimicrobial response and serves to disrupt the membrane of bacterial cells. Perforin creates pores in target cell membranes to facilitate entry of cytolytic molecules. IFNG and TNFA are inflammatory cytokines. TRAIL is a death receptor ligand that is highly expressed on NK cells where it is crucial for the anti-tumor response. IFNγ and TNFα are inflammatory cytokines.

In some cases, the genetically modified lymphocyte is modified in one or more of the following ways: (a) to reduce or prevent expression of endogenous human granzyme A, granzyme H, granzyme M, and/or tryptase-2; (b) to reduce or prevent expression of endogenous death receptor ligands TRAIL and FasL; and/or (c) to reduce or prevent secretion of inflammatory cytokines IFNγ and TNFα. To achieve reduced or knocked-out expression of the above proteins, the cell is modified such that one or more of its TRAIL, FASLG, IFNG, TNFA, GZMA, GZMH, GB/DI, and TPSB2 genes have been disrupted by genetic knockout. As a result of the genetic knockout, the genetically modified cell exhibits reduced expression of the corresponding endogenous gene product (e.g., human TRAIL, FasL IFNγ TNFα, granzyme A, granzyme H, granzyme M, and/or tryptase-2) relative to unmodified control cells.

In some embodiments, the genetically modified lymphocyte comprises one or more modified cytotoxic proteins that results in reduced or eliminated expression of the cytotoxic protein(s) in the lymphocyte. In further embodiments, the engineered lymphocytes may comprise two or more modified cytotoxic proteins, or alternatively three or more modified cytotoxic proteins. In some embodiments, the engineered lymphocyte comprises one or more mutated granzymes, two or more mutated granzymes, or three or more mutated granzymes, wherein the granzymes are mutated to reduce or eliminate expression within the cell. In some embodiments, the genetically modified lymphocytes comprise mutations in two or more cytotoxic proteins that reduce or eliminate the expression of the two or more cytotoxic proteins in the lymphocytes. For example, in some embodiments, the lymphocytes comprise mutations in 2, 3, 4, 5, 6, 7, 8, 9, or cytotoxic proteins. In the examples, the inventors generate triple knockout T cells that comprise mutations in three granzyme proteins (i.e., GZMA, GZMB, and GZMM). Thus, in some embodiments, the two or more cytotoxic proteins comprises GZMA, GZMB, and GZMM. In other embodiments, the genetically modified lymphocytes comprise mutations in all five human granzyme proteins (i.e., GZMA, GZMB, GZMH, GZMK, and GZMM).

As used herein, the term “reduced expression” refers to any reduction in the expression of an endogenous polypeptide in a genetically modified cell when compared to an unmodified control cell. The term reduced can also refer to a reduction in the percentage of cells in a population of cells that express an endogenous polypeptide when compared to a population of unmodified control cells. Such a reduction may be up to 5%, 10%, 20%, 30%, 40%, 50%, 60%, 70%, 80%, 90%, 95%, or up to 100%. Accordingly, the term “reduced” encompasses both a partial knockdown and a complete knockdown of the endogenous polypeptide.

As used herein, an “unmodified control cell” refers to a cell that of the genotype that was used as starting material for the generation of the genetically modified cell. For example, if the genetically modified cell was generated by introducing mutations into a wild-type gamma-delta T cell, the unmodified control cell should be a wild-type gamma-delta T cell. Thus, the unmodified control cell serves as a reference point for measuring changes in genotype or phenotype produced by genetically modifying the cell.

Reduced expression of cytotoxic proteins can be measured at the protein level, e.g., using flow cytometry, ELISA, ELISPOT, cytometric bead array or other multiplex methods, western blot and other immunoaffinity-based methods. Antibodies that bind to specific proteins are well-known in the art and some are commercially available, as are ELISA kits. Alternatively, expression of cytotoxic proteins can be measured at the RNA level, e.g., using RT-PCR or sequencing.

The “cargo molecule” may be any exogenous protein, nucleic acids, including an RNA molecule, DNA molecule, or small molecule that one wishes to deliver to a target cell. Suitable methods to deliver, produce or incorporate the cargo into the genetically modified lymphocyte such that it can be delivered to the target cell are known in the art. For example, a construct (e.g., plasmid, vector, or viral vectors) can be used to deliver a protein or nucleic acid into the genetically modified lymphocytes.

In some embodiments, the lymphocyte comprises a first construct encoding the cargo molecule operably linked to a promoter. As used herein, the term “construct” or “nucleic acid construct” refers to an artificially constructed (i.e., not naturally occurring) DNA molecule. In some embodiments, the cargo molecules may be a nucleic acid sequence or construct, e.g., a RNA molecule, DNA molecule, plasmid or vector that one may want to deliver to the target cell. In another embodiment, the cargo molecule may be a protein or mRNA and a construct or vector may be used to express the protein or mRNA within the genetically modified lymphocyte (e.g. construct encodes the mRNA or protein and comprises a sequence for the protein or mRNA operably linked to a promoter). As described more herein, the cargo molecule may be a fusion protein that comprises the cargo fused to a catalytically inactive granzyme. In some embodiments, the first construct encodes a fusion protein comprising a catalytically inactive granzyme fused to the cargo molecule protein and comprises a promoter that allows expression of the fusion protein within the lymphocyte.

In some embodiments, a construct is provided that comprises or consists of the polynucleotide encoding the cargo described herein and a heterologous sequence. Nucleic acid constructs may be part of a vector that is used, for example, to transform a cell. When referring to a nucleic acid molecule alone (as opposed to a viral particle, see below), the term “vector” is used herein to refer to a nucleic acid molecule capable of propagating another nucleic acid to which it is linked. The term includes the vector as a self-replicating nucleic acid structure that can be packaged into viral particles and can be expressed in dividing and non-dividing cells either extrachromosomally or integrated into the host cell genome. Certain vectors are capable of directing the expression of nucleic acids to which they are operatively linked. Within the construct, the polynucleotide may be operatively linked to a transcriptional promoter (e.g., a heterologous promoter) allowing the construct to direct the transcription of said polynucleotide in a host cell. Such vectors are referred to herein as “recombinant constructs,” “expression constructs,” “recombinant expression vectors” (or simply, “expression vectors” or “vectors”).

Suitable vectors are known in the art and contain the necessary elements in order for the gene encoded within the vector to be expressed as a mRNA or protein in the host cell. The term “vector” refers to a nucleic acid molecule capable of transporting another nucleic acid to which it has been linked. One type of vector is a “plasmid”, which refers to a circular double stranded DNA loop into which additional DNA segments may be ligated, specifically exogenous DNA segments encoding the antibodies or fragments thereof. Another type of vector is a viral vector, wherein additional DNA segments may be ligated into the viral genome. Certain vectors are capable of autonomous replication in a host cell into which they are introduced. Other vectors can be integrated into the genome of a host cell upon introduction into the host cell, and thereby are replicated along with the host genome (e.g. lentiviral vectors). Vector includes expression vectors, such as viral vectors (e.g., replication defective retroviruses (including lentiviruses), adenoviruses and adeno-associated viruses (rAAV)), which serve equivalent functions. Lentiviral vectors may be used to make suitable lentiviral vector particles by methods known in the art to transform cells in order to express the reporter described herein.

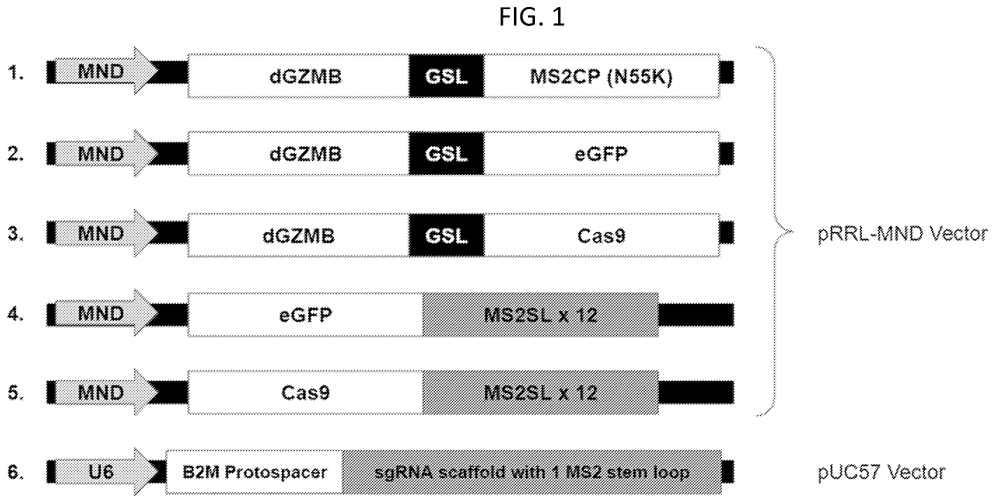

As used herein, the term “promoter” refers to a DNA sequence capable of controlling the expression of a coding sequence or functional RNA. In general, a coding sequence is located 3′ to a promoter sequence. Promoters may be derived in their entirety from a native gene, or may be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. Promoters that cause a gene to be expressed in most cell types at most times are commonly referred to as “constitutive promoters”, whereas promoters that allow the selective expression of a gene in most cell types are referred to as “inducible promoters”. In the examples, the inventors used the MND promoter and U6 promoter to drive the expression of cargo molecules in the genetically modified lymphocytes (see FIG. 1). Thus, in some embodiments, the promoter is the MND promoter, which is a constitutively active, synthetic promoter that contains the U3 region of a modified MoMuLV LTR with a myeloproliferative sarcoma virus enhancer. In other embodiments, the promoter is the U6 promoter, which is a type III promoter that drives expression of small RNAs. In other embodiments, the promoter is the endogenous promoter of the GZMB gene. However, other suitable promoters known and understood in the art may be used in the practice of the present invention. Examples of other suitable promoters include, without limitation, CMV, EF1A, CAGG, PGK, UBC, GAPDH, PPP1R12C (AAVS1), TRAC, and other endogenous promoters. In some embodiments, the genetically modified lymphocytes further comprise a chimeric antigen receptor. A “chimeric antigen receptor (CAR)” is an artificial T-cell receptor that gives T cells the new ability to target a specific antigen. CARs comprise an extracellular antigen binding domain operably linked to a transmembrane domain and at least one intracellular domain, which activates the T cell when an antigen is bound. Generally, the antigen binding domain of a CAR has specificity for a particular antigen expressed on the surface of a target cell of interest. For example, a lymphocyte can be genetically modified to express an anti-CD34 CAR that specifically binds to CD34, a marker of hematopoietic stem cells (HSCs). In such cases, when the modified lymphocyte is co-cultured with HSC target cells expressing CD34, the target cells will activate the lymphocyte via the anti-CD34 CAR. The lymphocyte will then form an immunological synapse with the target cell and release cytolytic granules, the contents of which will enter the target cell via a perforin pore. Other suitable surface markers of HSCs that can be used to specifically target HSCs with CAR-expressing modified lymphocytes include, without limitation, cKIT, CD90, and CD49f. As is described in the Examples, the inventors generated genetically modified T cells comprising a CAR that binds to the antigen CD19, which is commonly found on the surface of B cell tumors. Thus, in some embodiments, the CAR is an anti-CD19 CAR. An anti-CD19 CAR may comprise any antigen recognition domain that specifically binds to CD19. For example, in some embodiments, the antigen recognition domain comprises a single-chain variable fragment (scFv) antibody that binds to CD19. Thus, suitably, the genetically modified lymphocytes therefore comprise an exogenous nucleic acid construct that is capable of expression of the CAR within the lymphocyte.

In some embodiments, a catalytically inactive form of granzyme B (dGZMB) is used to chaperone the cargo molecule through the immunological synapse when the lymphocytes bind to the target cell. Granzyme B (GZB) is a serine protease that is loaded into cytotoxic granules and delivered to target cells as part of the granzyme-perforin pathway. Any catalytically inactive granzyme protein can be used with the present invention. For instance, an inactive granzyme B protein (dGZMB protein) can be obtained by mutating a serine amino acid in the active site of a wild-type GZBM protein to an alanine. In some embodiments, the dGZMB protein comprising a serine to alanine substitution at amino acid position 203. In other embodiments, the dGZMB protein comprising a serine to alanine substitution at amino acid position 195. In some embodiments, dGZMB is encoded by SEQ ID NO:12. It is contemplated that other catalytically inactive granzymes may also be used in the production of fusion proteins as described herein without deviating from the present invention.

In other embodiments, granulysin or a catalytically inactive granulysin is used to chaperone the cargo molecule through the immunological synapse. Granulysin (GNLY), which is an antimicrobial peptide expressed only by killer lymphocytes, selectively disrupts bacterial, fungal, and parasite membranes in infected immune cells via delivery of cytotoxic granules via cell-cell connections similar to the perforin-granzyme pathway. As reported by Crespo et al. (2020, Cell 182, 1-15), decidual NK (dNK) and peripheral NK (pNK) cells transfer GNLY to kill bacteria in cells types including macrophages, dendritic cells, and trophoblasts without killing the host cell. Unlike GZBM, GNLY would not require enzyme inactivating mutations to avoid killing the target immune cell.

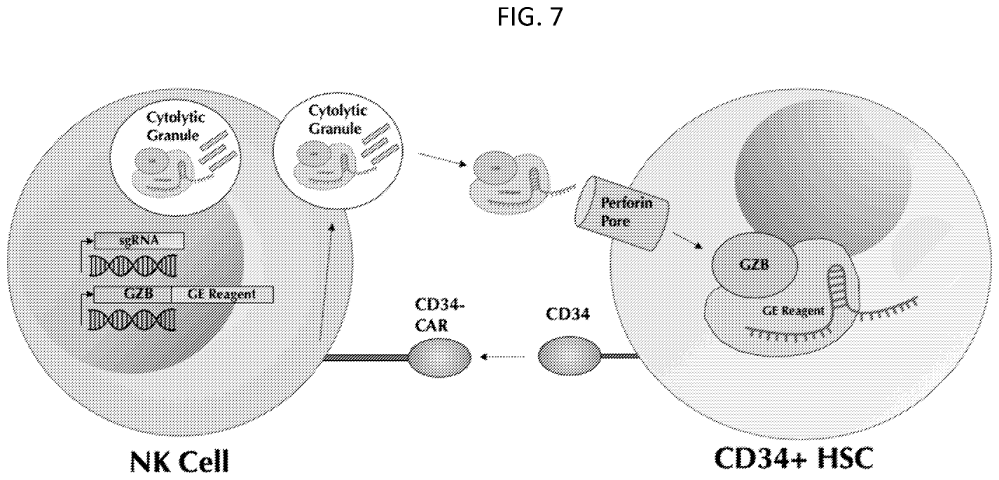

The inventors have generated constructs from which catalytically inactive granzyme (e.g. dGZMB) is expressed as a fusion protein with a cargo protein or is expressed as a fusion protein with a protein that binds to a cargo RNA. A “fusion protein” is a protein consisting of at least two domains that are encoded by separate genes that have been joined such that they are transcribed and translated as a single unit, producing a single polypeptide. For example, as is depicted in FIG. 7, dGZMB can be expressed as a fusion protein with a gene editing enzyme that binds to a guide nucleic acid (e.g., an sgRNA), such that dGZMB chaperones the whole gene editing complex into the target cell. Thus, in some embodiments, the cargo molecule is expressed as a fusion protein that comprises dGZMB. Conjugation of a cargo molecule to dGZMB should ensure that the cargo molecule is packaged into cytotoxic granules and delivered to the target cell along with dGZMB. Thus, in some embodiments, the cargo molecule is encapsulated in cytotoxic granules within the T-lymphocytes.

In some embodiments, the cargo molecule is linked to dGZMB by a linker within the fusion protein. As used herein, the term “peptide linker” or “linker” refers to a peptide sequence that bridges two protein segments. Ideally, the linker should facilitate a functional connection between the two protein segments (i.e., a connection that facilitates a degree of stability and proper folding of the protein segments into a three dimensional structure such that they perform some or all of biological activities of the proteins from which they are derived within the context of the fusion protein). The linker may “flexible” such that it has no required fixed structure in solution and the adjacent protein segments are free to move relative to one another. The flexible linker comprises 1 or more amino acid residues, preferably 1, 2, 3, 4, 5, 6, 7, 8, 9, 10, 11, 12, 13, 14, 15, 16, 17, 18, 19, or 20 or more residues. The linker may be an existing sequence provided by a protein included in the fusion protein or it may be provided by insertion of one or more amino acid residues between the peptides of the fusion protein. The linker may comprise any amino acid sequence that does not substantially hinder the interaction of the polypeptides with their corresponding target molecules. Preferred amino acid residues for flexible linker sequences include glycine, alanine, serine, threonine, lysine, arginine, glutamine and glutamic acid, but are not limited thereto. In some embodiments, the linker peptide is a glycine-serine linker (GSL). In some embodiments, the GSL is encoded by SEQ ID NO:13. For example, described herein is (1) a fusion protein comprising a MS2 cap protein (MS2CP) containing high affinity N55K mutation fused to dGZMB via a flexible GSL, and (2) a fusion protein comprising dGZMB fused to Cas9 via a flexible GSL. For each fusion protein, the preferred linker length will depend upon the nature of the polypeptides to be linked and the desired activity of the linked fusion polypeptide resulting from the linkage. Generally, the linker should be long enough to allow the resulting linked fusion polypeptide to properly fold into a conformation providing the desired biological activity.

In some embodiments, dGZMB is used to chaperone an RNA cargo molecule through the immunological synapse. In these embodiments, the lymphocyte further comprising a first construct encoding a fusion protein comprising dGZMB linked to an RNA-binding protein that binds the RNA cargo molecule, operably linked to a promoter and a second construct encoding the RNA cargo molecule.

Any RNA-binding protein that specifically binds to the RNA cargo molecule may be used with the present invention. For example, in some embodiments, the RNA cargo encoded by the first construct comprises MS2 stem loops, and the fusion protein encoded by the second construct comprises dGZMB and an MS2-binding protein, such that the fusion protein binds to the RNA cargo and chaperones it through the immunological synapse. For example, the inventors have generated a construct encoding a fusion protein comprising dGZMB and a modified version of the MS2 cap protein (MS2CP) (see construct #1 in FIG. 1, and SEQ ID NO:1). This modified MS2CP protein has an N55K amino acid substitution, which confers increased affinity for MS2 stem loops. In some embodiments, the MS2CP protein is encoded by SEQ ID NO:14. Thus, in some embodiments the RNA cargo molecule comprises a one or more MS2 stem loops. For example, the RNA cargo molecule may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more MS2 stem loops. Examples of RNA cargo molecules comprising 6, 12, and 24 MS2 stem loops can be found in the eGFP-MS2SL fusion proteins encoded by SEQ ID NOs: 4-6 (see construct #4 in FIG. 1). In some embodiments, the MS2 stem loops are encoded by a sequence selected from SEQ ID NOs:17-19. However, many other RNA-binding proteins are known in the art and may be used with the present invention. Examples of other suitable RNA-binding proteins include, without limitation, PB7, COM, and THA8L.

In some embodiments, the genetically modified lymphocytes are designed to deliver gene editing reagents to the target cell. Suitable gene editing reagents for use with the present invention include Cas enzymes, base editors, and other nucleases.

Suitable Cas nucleases include, without limitation, Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, and Csf4, as well as any chimeras, mutants, homologs, or orthologs thereof. In some embodiments, the Cas nuclease is derived from the CRISPR system of Streptococcus pyogenes (SP) or Staphylococcus aureus (SA). In some embodiments, the Cas nuclease is Cas9. In some embodiments, Cas9 is encoded by SEQ ID NO:16. A comprehensive review of the Cas protein family is presented in Haft et al. (2005) Computational Biology, PLoS Comput. Biol. 1:e60. At least 41 CRISPR-associated (Cas) gene families have been described to date.

As used herein, “base editors” are fusion proteins that comprise a Cas nickase domain or catalytically dead Cas protein fused to a deaminase. As in CRISPR-based gene editing, base editors are targeted to a specific gene sequences using a guide nucleic acid. However, unlike CRISPR, base editing does not generate double-stranded DNA breaks, making it a safer alternative to Cas nuclease-based methods. Instead, base editing uses the deaminase enzyme to modify a single base without altering the bases around it. There are two classes of base editors: cytosine base editors (CBEs) and adenine base editors (ABEs). CBEs comprise a cytidine deaminase that converts cytidine to uridine within a small editing window near the protospacer adjacent motif (PAM) site. Uridine is subsequently converted to thymidine through base excision repair, creating a cytosine (C) to thymine (T) change (i.e., a guanosine to adenine change on the opposite strand). ABEs comprise an adenine deaminase, which creates an adenine (A) to guanosine (G) change. When a CBE is utilized, to prevent cells from repairing the modified base and encourage the cell to use the edited strand as a template for mismatch repair, a uracil DNA glycosylase inhibitor (UGI) is used to block base excision repair. In some embodiments, a UGI domain is included as part of the base editor fusion protein. In other embodiments, the UGI domain is provided to the cell as a separate component. Researchers have developed third and fourth generation base editors with improved efficiency. For example, the third generation CBE base editor BE3 (i.e., base editor 3) uses a Cas9 nickase to nick the unmodified DNA strand so that it appears “newly synthesized” to the cell, forcing the cell to repair the DNA using the deaminated strand as a template, whereas fourth generation base editors systems (i.e., base editor 4 (BE4)) employ two copies of base excision repair inhibitor UGI. In some embodiments, a BE3 or BE4 cytosine base editor is used in the methods of the present invention. In other embodiments, a CBE comprising a different deaminase, such as hA3A-BE4, hA3G-BE4, evoFERNY-BE4, or evoCDA-BE4, is used. In other embodiments, an ABE base editor, such as ABE6.3, ABE7.10, ABE8e, or ABE8.20 is used. In some embodiments, the base editor enzymes are mutated or modified to confer a desired functionality such as reduced guide-independent off-target editing, reduced guide-dependent off-target editing, an altered editing window, an altered editing context preference, an altered target site specificity, or more precise target editing.

Other suitable nucleases for use with the present invention include, without limitation, hADARdE>Q, APOBEC cytidine deaminase, MutY DNA glycosylase, or apurinic endonuclease.

For example, in some embodiments, the cargo molecule is a Cas9 protein, an RNA encoding Cas9, a guide nucleic acid (gNA) or combinations thereof. In the examples, the inventors generated constructs encoding these cargo molecules. Specifically, they generated constructs encoding (1) a fusion protein comprising dGZMB fused to Cas9 via a flexible glycine serine linker (i.e., dGZMB-GSL-Cas9, see construct #3 in FIG. 1, and SEQ ID NO:3), (2) an RNA encoding Cas9 fused to a series of 24 MS2 stem loops (i.e., Cas9-MS2SLx24, which is similar to construct #5 in FIG. 1, see SEQ ID NO:7), and (3), a guide RNA targeting the beta-2-microglobulin (B2M) locus that includes one MS2 stem loop within its gRNA scaffold (i.e., B2MsgRNA-MS2SL, see construct #4 in FIG. 1, see SEQ ID NO:8).

In a second aspect, the present invention provides methods for making the genetically modified lymphocytes described herein. The methods comprise, at a minimum: (a) introducing into the lymphocyte gene editing reagents comprising a gene editing nuclease and one or more guide nucleic acid (gNA) that targets one or more gene encoding a cytotoxic protein; and (b) introducing into the lymphocyte a construct encoding a cargo molecule operably linked to a promoter. In these methods, the gene editing reagents generate a mutation in one or more cytotoxic protein that reduces or eliminates the expression of the cytotoxic protein in the lymphocytes, and the resulting lymphocytes express the cargo molecule and encapsulate it within cytotoxic granules.

The “gene editing reagents” used in the methods of the present invention include a gene editing enzyme and one or more gNAs. As used herein, the term “gene editing enzyme” refers to an enzyme that can be used to make a targeted mutation in a genome. Suitable gene editing enzymes for use with the present invention include the Cas enzymes, base editors, and other nucleases described in the section above. In some embodiments, the gene editing enzyme is Cas9.

“Guide nucleic acids (gNAs)” are nucleic acids that function as guides for gene editing enzymes, which they form complexes with. Any appropriate gNA can be used in the present methods. The particular gNA sequence(s) will depend on the target DNA sequence to be edited in the lymphocyte genome. In some cases, a gNA comprises a sequence of at least 10 contiguous nucleotides, and often a sequence of 17-23 or 18-22 contiguous nucleotides, that is complementary the target DNA sequence. In some embodiments, a gNA is from 20 to 300 or more bases in length, or more. In certain embodiments, a gNA is from 20 to 300 bases in length, or 20 to 120 bases, or 30 to 50 bases, or 39 to 46 bases in length. The gNAs may comprise a nucleotide sequence that is partially or wholly complementary to the target DNA sequence. The target DNA sequence should be selected such that there is an appropriate (i.e., recognized by the gene editing enzyme) protospacer adjacent motif (PAM) located immediately downstream from the target site. Examples of PAM sequence are known (see, e.g., Shah et al., RNA Biology 10 (5): 891-899, 2013). In some embodiments, the gNA is a single guide RNA (sgRNA). In some cases, the gNA is expressed from a plasmid or a viral vector, or is delivered to a cell as a nucleic acid. As used herein, the terms “complementary” or “complementarity” are used in reference to “polynucleotides” and “oligonucleotides” (which are interchangeable terms that refer to a sequence of nucleotides) related by the base-pairing rules. For example, the sequence “5′-C-A-G-T,” is complementary to the sequence “5′-A-C-T-G” Complementarity can be “partial” or “total.” “Partial” complementarity is where one or more nucleic acid bases is not matched according to the base pairing rules. “Total” or “complete” complementarity between nucleic acids is where each and every nucleic acid base is matched with another base under the base pairing rules.

As used herein, the term “complementary” refers to the ability of a nucleic acid molecule to bind to (i.e., hybridize with) another nucleic acid molecule through the formation of hydrogen bonds between specific nucleotides (i.e., A with T or U and G with C), forming a double-stranded molecule. Examples of sgRNAs that can be used to knock out GZMA, GZMB, and GZMIM are provided as SEQ ID NOs:9-11 (see Table 1).

In some cases, it may be advantageous to use chemically modified gNAs having increased stability when transfected into mammalian cells. For example, gNAs can be chemically modified to comprise 2′-O-methyl phosphorthioate modifications on at least one 5′ nucleotide and at least one 3′ nucleotide. In some cases, the three terminal 5′ nucleotides and three terminal 3′ nucleotides are chemically modified to comprise 2′-O-methyl phosphorthioate modifications.

The terms “nucleic acid” and “nucleic acid molecule,” as used herein, refer to a compound comprising a nucleobase and an acidic moiety, e.g., a nucleoside, a nucleotide, or a polymer of nucleotides. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g. nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or include non-naturally occurring nucleotides or nucleosides. Furthermore, the terms “nucleic acid,” “DNA,” “RNA,” and/or similar terms include nucleic acid analogs, i.e. analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadeno sine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

The terms “protein,” “peptide,” and “polypeptide” are used interchangeably herein and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof. A protein may comprise different domains, for example, a nucleic acid binding domain and a nucleic acid cleavage domain. In some embodiments, a protein comprises a proteinaceous part, e.g., an amino acid sequence constituting a nucleic acid binding domain, and an organic compound, e.g., a compound that can act as a nucleic acid cleavage agent.

Nucleic acids, proteins, and/or other molecules of this disclosure may be purified. As used herein, purified means separate from the majority of other compounds or entities. A compound or moiety may be partially purified or substantially purified. Purity may be denoted by a weight by weight measure and may be determined using a variety of analytical techniques such as but not limited to mass spectrometry, HPLC, etc.

Nucleic acids, proteins, and/or other molecules of this disclosure may be isolated. As used herein, “isolated” means to separate from at least some of the components with which it is usually associated whether it is derived from a naturally occurring source or made synthetically, in whole or in part.

The lymphocytes of the present invention are genetically modified to reduce or eliminate expression of one or more cytotoxic protein. In other words, the lymphocytes comprise mutations that “knock out” one or more cytotoxic protein. Knockout mutations can be generated using any suitable gene editing method. For example, knockouts can be generated using traditional CRISPR-Cas9 indel formation or using base editors to disrupt splice site acceptors (e.g., to cause out of frame translation or introduce a premature STOP codon).

Additionally, the lymphocytes of the present invention are modified to introduce a cargo molecule. In some embodiments, the cargo molecule is a gene editing reagent.

Constructs for knocking out the cytotoxic protein(s) and the constructs encoding cargo molecules may be introduced into the lymphocytes using any suitable gene delivery method, including a vector-based system. Preferably, the vector is a viral vector, such as a lenti- or baculo- or preferably adeno-viral/adeno-associated viral (AAV) vector, but other means of delivery are known (such as yeast systems, microvesicles, liposomes, gene guns/means of attaching vectors to gold nanoparticles) and are contemplated. In other cases, the cells are simply electroporated for uptake of constructs, nucleic acids, or proteins.

In some embodiments, the methods further comprise (1) introducing into the lymphocyte a construct encoding a CAR, (2) introducing into the lymphocyte a construct encoding a fusion protein comprising dGZMB linked to an RNA-binding protein that binds an RNA cargo molecule, and/or (3) introducing into the lymphocytes a second construct encoding the RNA cargo molecule.

The genetically modified lymphocytes comprise mutations in one or more gene encoding a cytotoxic protein. Knockout of multiple target genes may be accomplished via multiplexed gene editing of primary human lymphocytes. In other cases, the engineered lymphocytes are obtained by gene editing pluripotent stem cells and then differentiating the genetically modified pluripotent cells in vitro to obtain the differentiated lymphocyte cell type of interest. Exemplary protocols for directing differentiation of pluripotent stem cells to NK cells are described, for example, in Li et al. (Cell Stem Cell 23, 181-192.e5 (2018)) and Shankar et al. (Stem Cell Res Ther 11, (2020)), each of which is incorporated herein by reference. Exemplary protocols for directing differentiation of pluripotent stem cells to T cells are described, for example, in Iriguchi et al. (Nat Commun. 12 (1):430 (2021)) and Trotman-Grant et al. (Nat Commun. 12 (1):5023 (2021)).

Any appropriate method can be performed to confirm successful gene editing. For instance, the genetic locus targeted for gene editing can be sequenced to determine whether editing occurred. Alternatively, if gene editing was performed to alter the expression of a protein or RNA, detection of protein or RNA expression can be used to confirm successful gene editing.

In some cases, engineered lymphocytes of this disclosure have been modified to comprise one or more nucleic acids encoding a (i) fusion protein comprising granulysin or a catalytically inactive granulysin fused to cargo molecules, optionally a gene editing reagent, for example, Cas9 nuclease domain or a MS2 RNA binding protein; and (ii) one or more gNAs having complementarity to a target nucleic acid sequence to be genetically modified in a target cell. The human lymphocyte cell can be further genetically modified to express a chimeric antigen receptor (CAR) comprising a single-chain variant fragment (scFv) specific for the target cell, whereby the herein said genetically-modified human lymphocyte cell expresses said CAR.

### Methods for Delivery Cargo to a Target Cell

In a third aspect, the present invention provides methods for delivering a cargo molecule to a target cell, which can be done in vitro or in vivo. In some aspects, provided herein are method for in vivo delivery of molecules (e.g., gene editing reagents, therapeutic payload) to a target cell. Unlike conventional methods, which use viruses or engineered polymers to achieve in vivo delivery, the methods of this disclosure use genome-engineered cells that can traverse the human body, identify specific cell types, and deliver molecules to target cells. It is challenging to be selective about which cells receive molecules when using viruses or polymers, and efficiency can be very low. Accordingly, the methods of this disclosure provide for selective, targeted delivery of molecules in the in vivo setting and, thus, are advantageous over conventional methods. The methods comprise co-culturing the target cell with a genetically modified lymphocyte described herein, such that the lymphocyte is activated by the target cells and the cargo molecule is delivered to the target cell. In some embodiments, the co-culturing is in vivo, and therefore the co-culturing comprises contacting (e.g., administering) the lymphocytes to a subject allowing for the lymphocytes to locate and dock with the target cells in vivo.

The methods of the present invention may be used to target cargo molecules to any target cell. In some cases, the target cell is an immune cell (e.g., a T cell, B cell, tumor-infiltrating lymphocyte (TIL), or dendritic cell). Other target cell types include, without limitation, CD34+ hematopoietic stem cells (HSCs), tumor cells, hepatocytes, liver stellate cells, aortic smooth muscle cells, cardiac myocytes, neural cells (e.g., neurons, macroglia, microglia), fibroblasts, keratinocytes (e.g., LGR5+ keratinocytes), epithelial cells, hair follicle stem cells, and muscle cells and progenitors thereof (e.g., PAX7+ muscle stem cells).

In some embodiments, the genetically modified lymphocyte used in the method comprises a CAR. The CAR may comprise a ligand-binding domain that specifically binds to an antigen present on the surface of the target cell. In the Examples, the inventors generated genetically modified lymphocytes that comprise an anti-CD19 CAR such that they target CD19-expressing cells. CD19 is an antigen that is commonly expressed on the surface of B cell tumors. Thus, in some embodiments, genetically modified lymphocytes comprise a CAR that specifically binds to CD19 and the target cell is a cancer cell.

Any appropriate method can be performed to confirm successful delivery of granzyme B fusion proteins or cargo to the target cell. For instance, when the fusion protein comprises gene editing reagents, sequencing of the genetic locus targeted for gene editing can be sequenced to determine whether editing occured. Effects of gene editing or modulation by introduction of therapeutic cargo can be determined by any appropriate methods. For example, methods and techniques for assessing the expression and/or levels of cell markers are known in the art. Antibodies and reagents for detection of such markers are well known in the art, and readily available. Assays and methods for detecting such markers include, but are not limited to, flow cytometry, including intracellular flow cytometry, ELISA, ELISPOT, cytometric bead array or other multiplex methods, western blot and other immunoaffinity-based methods.

In some embodiments, the methods are used to treat a disease in a subject. In these embodiments, co-culturing of the target cell with the genetically modified lymphocyte may be accomplished (1) in vivo by administering the genetically modified lymphocyte to the subject or (2) ex vivo by removing a target cell from the subject for co-culture with the genetically modified lymphocyte and then returning the cells to the subject as an autologous transplant.

As used herein, the term “administering” refers to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, intradermal administration, intrathecal administration, and subcutaneous administration. Administration can be continuous or intermittent. In some embodiments, the genetically modified lymphocyte is administered as part of a composition comprising a therapeutically effective amount of the genetically modified lymphocyte and a pharmaceutically acceptable carrier.

The term “therapeutically effective amount” refers to an amount sufficient to effect beneficial or desirable biological or clinical results. That result can be reducing, alleviating, inhibiting or preventing one or more symptoms of a disease or condition, reducing, inhibiting or preventing the growth of cancer cells, reducing, inhibiting or preventing metastasis of the cancer cells or invasiveness of the cancer cells or metastasis, or reducing, alleviating, inhibiting or preventing one or more symptoms of the cancer or metastasis thereof, or any other desired alteration of a biological system.

“Pharmaceutically acceptable carriers” are known in the art and include, but are not limited to, diluents (e.g., Tris-HC1, acetate, phosphate), preservatives (e.g., Thimerosal, benzyl alcohol, parabens), solubilizing agents (e.g., glycerol, polyethylene glycerol), emulsifiers, liposomes, nanoparticles, and adjuvants. Pharmaceutically acceptable carriers may be aqueous or non-aqueous solutions, suspensions, or emulsions. Examples of nonaqueous solvents are propylene glycol, polyethylene glycol, vegetable oils such as olive oil, and injectable organic esters such as ethyl oleate. Aqueous carriers include isotonic solutions, alcoholic/aqueous solutions, emulsions or suspensions, including saline and buffered media.

The methods can be used to deliver various therapeutic payloads to target cells. For example, the methods can be used to deliver cytotoxic agents to treat cancer, pro-survival factors to treat degenerative diseases, or enzymes deficient in a subject to treat metabolic diseases. As one specific example, liver cells can be gene edited to restore synthesis of lysosomal enzyme α-1-iduronidase (IDUA), which is deficient in patients with the genetic disorder mucopolysaccharidosis type I (MPS I). As another specific example, immune cells such as TIL can be targeted in vivo to disrupt intracellular checkpoint genes. Further, skin cells such as LGR5+ keratinocytes can be targeted to correct recessive epidermolysis bullosa or junctional epidermolysis bullosa (JEB).

In some embodiments, the methods are used to treat a cancer or precancerous condition. The cancer may include, for example, bone cancer, brain cancer, breast cancer, cervical cancer, cancer of the larynx, lung cancer, pancreatic cancer, prostate cancer, skin cancer, cancer of the spine, stomach cancer, uterine cancer, hematopoietic cancer, and/or lymphoid cancer, etc. A hematopoietic cancer and/or lymphoid cancer may include, for example, acute myelogenous leukemia (AML), acute lymphoblastic leukemia (ALL), myelodysplastic syndromes (MDS), non-Hodgkin lymphoma (NHL), chronic myelogenous leukemia (CIVIL), Hodgkin's disease, and/or multiple myeloma. The cancer may be a metastatic cancer. The precancerous condition can be a preneoplastic lesion.

In some embodiments, the methods are used to treat a monogenic disease. A monogenic disease is a disease caused by a variation (e.g., a single nucleotide polymorphism) in a single gene. In these embodiments, gene editing reagents (e.g., a base editor and a sgRNA) are targeted to cells containing the variation, such that the gene variation is edited to treat the disease.

### Additional Aspects

In another aspect, the disclosure provides methods of genetically modifying a target cell. The methods comprise contacting the target cell with a genetically modified lymphocyte described herein. In these embodiments, the cargo molecule is a gene editing reagent, such as a protein (e.g., a Cas9 protein) or an RNA encoding Cas9, a guide nucleic acid (gNA), or combinations thereof. In some embodiments, the genetically modified lymphocyte expresses Cas9 nuclease protein and at least one guide RNA and activation of the lymphocyte via interaction with a target cell initiates transfer of the gene editing protein and at least one guide RNA to the target cell. In some embodiments, the methods further comprise incubating the cells for a sufficient time for the target cell to be genetically modified by the gene editing reagents. Suitably, the genetically modified lymphocyte expresses a targeting protein (e.g., a CAR) that targets it to the target cell.

In another aspect, the disclosure provides methods for delivering one or more gene editing reagent to a target cell. The methods comprise: (a) providing a genetically modified human lymphocyte comprising one or more (i) a cargo molecule comprising a gene editing reagent (e.g., Cas9 nuclease or base editor protein), optionally, in some embodiments, where the gene editing reagent is a fusion protein comprising a catalytically dead granzyme (dGZ) fused to gene editing reagent, and one or more gNAs having complementarity to a target nucleic acid sequence to be genetically modified in the target cell, and/or (iii) a targeting molecule, preferably a chimeric antigen receptor (CAR), wherein the targeting molecule comprises a ligand-binding domain having specificity for an antigen present on the target cell, whereby a genetically modified human lymphocyte that expresses at last one cargo, a targeting molecule or both is obtained. In another aspect, the genetically modified human lymphocyte comprises (ii) a cargo molecule comprising a RNA biding protein, for example, a fusion protein comprising dGZ and a MS2 RNA-binding protein, and RNA one wishes to target to the target cell (e.g. the RNA may be encoded within a second construct and have one or more MS2 stem loops). The method may further comprise (b) incubating the genetically modified lymphocyte with the target cell under conditions that promote activation of the genetically modified lymphocyte and delivery of the cargo (e.g., one or more nucleic acids or proteins) to the target cell, preferably through perforin-induced membrane pores. As described herein, the lymphocyte may be further modified (a) to reduce or prevent expression of endogenous human granzyme A, granzyme H, granzyme M, tryptase-2, or combinations thereof; (b) to reduce or prevent expression of endogenous death receptor ligands TRAIL and FasL; (c) to reduce or prevent secretion of inflammatory cytokines IFNγ and TNFα; or (d) combinations of (a)-(c).

In another aspect, the disclosure provides methods for delivering one or more gene editing reagent to a target cell. The methods comprise: (a) providing a genetically modified human lymphocyte comprising one or more exogenous nucleic acids encoding (i) a cargo molecule comprising a gene editing reagent, optionally in some embodiments where the gene editing reagent is a fusion protein comprising a catalytically dead granzyme (dGZ) fused to gene editing reagent, preferably a Cas9 nuclease or base editor protein, and one or more gNAs having complementarity to a target nucleic acid sequence to be genetically modified in the target cell; or (ii) a cargo molecule comprising a fusion protein comprising dGZ and a MS2 RNA-binding protein, and a second construct encoding an RNA and one or more MS2 stem loops; and/or (iii) a targeting molecule, preferably a chimeric antigen receptor (CAR), wherein the targeting molecule comprises a ligand-binding domain having specificity for an antigen present on the target cell, whereby a genetically modified human lymphocyte that expresses at last one cargo, a targeting molecule or both is obtained; and (b) incubating the genetically modified lymphocyte with the target cell under conditions that promote activation of the genetically modified lymphocyte and delivery of the one or more nucleic acids or proteins to the target cell, preferably through perforin-induced membrane pores. As described herein, the lymphocyte may be further modified (a) to reduce or prevent expression of endogenous human granzyme A, granzyme H, granzyme M, tryptase-2, or combinations thereof; (b) to reduce or prevent expression of endogenous death receptor ligands TRAIL and FasL; (c) to reduce or prevent secretion of inflammatory cytokines IFNγ and TNFα; or (d) combinations of (a)-(c).

In another aspect, a genetically modified human lymphocyte capable of gene editing a target cell is provided. The genetically engineered lymphocyte comprises (i) a cargo molecule or one or more nucleic acids encoding a cargo molecule, for example, the cargo molecule comprising a gene editing reagent, (e.g., a fusion protein comprising a catalytically dead granzyme B (dGZMB), granulysin, or catalytically inactive granulysin fused to a gene editing reagent, preferably Cas9 nuclease domain, or dGZMB fused to an MS2 RNA-binding protein, and (ii) one or more gNAs having complementarity to a target nucleic acid sequence to be genetically modified in a target cell. The human lymphocyte can be further genetically modified to express a chimeric antigen receptor (CAR) comprising a single-chain variant fragment (scFv) specific for the target cell. In some embodiments, the human lymphocyte is further modified (a) to reduce or prevent expression of endogenous human granzyme A, granzyme H, granzyme M, tryptase-2, or combinations thereof; (b) to reduce or prevent expression of endogenous death receptor ligands TRAIL and FasL; and/or (c) to reduce or prevent secretion of inflammatory cytokines IFNγ and TNFα. In some embodiments, the one or more of TRAIL, FASLG, IFNG, TNFA, GZMA, GZMH, GEVIJII, and TPSB2 genes have been disrupted by genetic knockout, wherein the genetically modified lymphocyte exhibits reduced expression of endogenous human granzyme A, granzyme H, granzyme M, tryptase-2, TRAIL, FasL or combinations thereof, and optionally further exhibits reduced secretion of IFNγ, TNFα or both relative to unmodified control cells.

In some embodiments, the cargo molecule or components of a construct described herein can be delivered to a cell in vitro, ex vivo, or in vivo. In some cases, a viral or plasmid vector system is employed for delivery of base editing components described herein. Preferably, the vector is a viral vector, such as a lenti- or baculo- or preferably adeno-viral/adeno-associated viral (AAV) vectors, but other means of delivery are known (such as yeast systems, microvesicles, gene guns/means of attaching vectors to gold nanoparticles) and are contemplated. In certain embodiments, nucleic acids encoding gNAs and base editor fusion proteins are packaged for delivery to a cell in one or more viral delivery vectors. Suitable viral delivery vectors include, without limitation, adeno-viral/adeno-associated viral (AAV) vectors, lentiviral vectors. In some cases, non-viral transfer methods as are known in the art can be used to introduce nucleic acids or proteins in mammalian cells. Nucleic acids and proteins can be delivered with a pharmaceutically acceptable vehicle, or for example, encapsulated in a liposome. Other means of delivery are known (such as yeast systems, microvesicles, gene guns/means of attaching vectors to gold nanoparticles) and are contemplated. In some cases, cells are electroporated for uptake of gNA and base editor. In some cases, DNA donor template is delivered as Adeno-Associated Virus Type 6 (AAV6) vector by addition of viral supernatant to culture medium after introduction of the gNA, Cas enzyme or base editor, and vector by electroporation.

Any appropriate method can be performed to confirm successful delivery of granzyme B fusion proteins and cargo to the target cell. For instance, when the fusion protein comprises gene editing reagents, sequencing of the genetic locus targeted for gene editing can be sequenced to determine whether editing occured. Effects of gene editing or modulation by introduction of therapeutic cargo can be determined by any appropriate methods. For example, methods and techniques for assessing the expression and/or levels of cell markers are known in the art. Antibodies and reagents for detection of such markers are well known in the art, and readily available. Assays and methods for detecting such markers include, but are not limited to, flow cytometry, including intracellular flow cytometry, ELISA, ELISpot, cytometric bead array or other multiplex methods, Western Blot, and other immunoaffinity-based methods.

Although constructs encoding human proteins are described herein, those of skill in the art will appreciate that non-human and/or synthetic amino acid sequences can be used in place of human amino acid sequences. It will also be appreciated that amino acid analogs can be inserted or substituted in place of naturally occurring amino acid residues. As used herein, the term “amino acid analog” refers to amino acid-like compounds that are similar in structure and/or overall shape to one or more of the twenty L-amino acids commonly found in naturally occurring proteins. Amino acid analogs are either naturally occurring or non-naturally occurring (e.g. synthesized). If an amino acid analog is incorporated by substituting natural amino acids, any of the 20 amino acids commonly found in naturally occurring proteins may be replaced. While amino acids can be replaced (substituted) with amino acid analogs, in some cases amino acid analogs are inserted into a protein. For example, a codon encoding an amino acid analog can be inserted into the polynucleotide encoding the protein.

As used herein, “modifying” (“modify”) one or more target nucleic acid sequences refers to changing all or a portion of a (one or more) target nucleic acid sequence and includes the cleavage, introduction (insertion), replacement, and/or deletion (removal) of all or a portion of a target nucleic acid sequence. All or a portion of a target nucleic acid sequence can be completely or partially modified using the methods provided herein. For example, modifying a target nucleic acid sequence includes replacing all or a portion of a target nucleic acid sequence with one or more nucleotides (e.g., an exogenous nucleic acid sequence) or removing or deleting all or a portion (e.g., one or more nucleotides) of a target nucleic acid sequence. Modifying the one or more target nucleic acid sequences also includes introducing or inserting one or more nucleotides (e.g., an exogenous sequence) into (within) one or more target nucleic acid sequences.

It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. The term “consisting essentially of” and “consisting of” should be interpreted in line with the MPEP and relevant Federal Circuit interpretation. The transitional phrase “consisting essentially of” limits the scope of a claim to the specified materials or steps “and those that do not materially affect the basic and novel characteristic(s)” of the claimed invention. “Consisting of” is a closed term that excludes any element, step or ingredient not specified in the claim. For example, with regard to sequences “consisting of” refers to the sequence listed in the SEQ ID NO. and does refer to larger sequences that may contain the SEQ ID as a portion thereof.

As used in this specification and the claims, the singular forms “a,” “an,” and “the” include plural forms unless the context clearly dictates otherwise. For example, the term “a substituent” should be interpreted to mean “one or more substituents,” unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

The following Examples are offered for illustrative purposes only, and are not intended to limit the scope of the present invention in any way. Indeed, various modifications of the invention in addition to those shown and described herein will become apparent to those skilled in the art from the foregoing description and the following examples and fall within the scope of the appended claims.

### EXAMPLES

### Example 1

### Genetically Modified NK Cells

In a first experiment, GFP constructs are introduced into NK cell lines (e.g., NK92 cells or YT-Indy cells). The modified NK cells are co-cultured with target cells (e.g., K562 cells) and assayed for the presence of GFP in the target cells as proof of principle. K562 cells are a human immortalized myelogenous leukemia cells that are commonly used as target cells for NK assays due to their high, reproducible sensitivity to NK lysis.

In a second experiment, Cas9/gRNA constructs are introduced into NK cell lines (e.g., NK92 cells or YT-Indy cells). The modified NK cells are then co-cultured with target cells (e.g., K562 cells), and the target cells are assayed for the presence of gene editing.

Cell culture—NK92 and YT-Indy cells are cultured in RPMI 1640 medium supplemented with 20% FBS (fetal bovine serum), 1× GlutaMax, 1 mM sodium pyruvate, 10 mM HEPES, and 0.1 mM beta-mercaptoethanol. K562 cells are cultured in RPMI 1640 medium supplemented with 10% FBS.

Transfection—NK92 and YT-Indy cells are electroporated using the Thermo Fisher Neon system. 3e6 cells are washed once in PBS and resuspended in electroporation buffer R with 10-20 μg plasmid DNA. Cells+plasmid DNA will be electroporated according to the manufacturer's instructions with 3×10 ms pulses at 1250 V.

GFP co-culture assay—NK cell lines are transfected with GFP fusion constructs: dGZMB-GSL-GFP, dGZMB-GSL-MS2CP+EGFP-MS2SLx6, dGZMB-GSL-MS2CP+EGFP-MS2SLx12, dGZMB-GSL-MS2CP+EGFP-MS2SLx24. 48 hours after transfection, the transfected cells are analyzed for GFP expression by flow cytometry and enriched if necessary. 72 hours after transfection, NK cell lines are co-cultured at 37° C. with CellTrace Violet-labeled K562 at effector-to-target (E:T) ratios of 3:1, 1:1, and 1:3 for 2 hours, 6 hours, or overnight. Co-cultures are analyzed for GFP and CellTrace Violet expression by flow cytometry.

CRISPR co-culture assay—NK cell lines are transfected with Cas9/sgRNA fusion constructs: dGZMB-GSL-Cas9, dGZMB-GSL-Cas9+U6-B2MsgRNA-MS2SL, dGZMB-GSL-MS2CP+Cas9-MS2SLx24+U6-B2MsgRNA-MS2SL. 48 hours after transfection, the transfected cells are analyze for Cas9 expression by Western blot. 72 hours after transfection, NK cell lines are co-cultured at 37° C. with CellTrace Violet-labeled K562 at effector-to-target (E:T) ratios of 3:1, 1:1, and 1:3 for 2 hours, 6 hours, or overnight. CellTrace Violet-labeled K562 cells are sorted from the co-culture. After 7 days, K562 cells are analyzed for gene (e.g., B2M) knockout by flow cytometry and Sanger sequencing.

### Example 2

### Genetically Engineered T Cells

The following example demonstrates (1) that lymphocytes can be engineered to decrease their cytotoxic effects on target cells, and (2) that such engineered lymphocytes can be used to deliver protein or RNA cargo to a target cell through the immunological synapse.

### Construct Generation

Five constructs were made using a pRRL-MND plasmid backbone, which encodes LTRs that allow for lentivirus production of the construct (see constructs 1-5 in FIG. 1). Expression from these constructs is driven by MND, a synthetic promoter that is constitutively active, especially in hematopoietic cells. Constructs 1-3 encode a catalytically inactive form of granzyme B (dGZMB) comprising an S195A mutation followed by a flexible glycine-serine linker (GSL). Construct 1 encodes MS2 cap protein (MS2CP), which binds to RNA MS2 stem loops (MS2SL). Within this construct, MS2CP is included in the same open reading frame (ORF) as dGZMB-GSL and has an amino acid substitution of lysine in place of asparagine at position 55, which confers increased affinity of the cap protein for the MS2 stem loops. Construct 2 encodes dGZMB linked to enhanced green fluorescent protein (eGFP) via a GSL linker. This construct will be used to monitor transfer of protein through the immunological synapse. Construct 3 encodes dGZMB linked to Cas9 via a GSL linker. This construct will be used to examine the capabilities of this system to transfer large base editing proteins through the immunological synapse. Construct 4 encodes eGFP fused to a series of 12 MS2 stem loops, which contain repeats of 2 alternate binding sites. Construct 5 encodes Cas9 fused to the same MS2 stem loop sequence. Constructs 4 and 5 will be bound by the protein product of construct 1, and will be used to monitor transfer of mRNA into the target cells via binding to this protein. Notably, it may be possible to increase MS2 binding to constructs 4 and 5 by increasing the number of stem loops included in these constructs from 12 to 24 repeats. A sixth construct was made using the pUC57 plasmid backbone. This construct contains a U6 promoter, which is a type III promoter that drives expression of small RNAs. This construct encodes a single guide RNA (sgRNA) that targets the beta-2-microglobulin (B2M) locus via its protospacer sequence and includes one MS2 stem loop within its gRNA scaffold.

### Basal Expression of Cytotoxic Proteins

To engineer T cells with reduced cytotoxic effects, we first identified immunoregulatory genes that are highly expressed during T cell activation, allowing us to prioritize these genes for knockout. The following proteins of interest were selected for analysis based upon their known cytotoxicity and/or upregulation in immune cells (i.e., T cells and NK cells): granzyme A (GZMA), granzyme B (GZMB), granzyme K (GZMK), granzyme M (GZMM), Fas ligand (FasL), granulysin (GNLY), perforin, and tumor necrosis factor-related apoptosis-inducing ligand (TRAIL). Granzymes A, B, K, and M are serine proteases that induce caspase cleavage and trigger apoptosis in target cells. FasL is a ligand that is expressed on activated T cells and induces apoptosis of cells displaying the Fas receptor. GNLY is expressed by T cells during antimicrobial response and serves to disrupt the membrane of bacterial cells. Perforin creates pores in target cell membranes to facilitate entry of cytolytic molecules. TRAIL is highly expressed on NK cells where it is crucial for the anti-tumor response.

To quantify the expression of these proteins during T cell activation/expansion, CD3+ T cells from 4 healthy donors were cultured in T cell base media (OpTmizer CTS Basal Media+OpTmizer CTS Supplement, CTS Immune Cell SR, L-Glutamine, Penicillin/Streptomycin, N-Acetylcysteine) with IL-2, IL-7, and IL-15 and stimulated for T Cell expansion and activation using Dynabeads™ Human T-Activator CD3/CD28. Approximately 1,000,000 cells were collected on days 0, 1, 3, and 7 of activation/expansion and were stained with antibodies and subjected to flow cytometry. The following antibodies were used to stain the proteins of interest: Pacific Blue™ anti-human Granzyme A [Clone: CB9], Alexa Fluor® 700 anti-human/mouse Granzyme B Recombinant [Clone: QA16A02], PE/Cyanine7 anti-human Granzyme K [Clone: GM26E7], Granzyme M Mouse anti-Human, PE, Clone: 4B2G4, Invitrogen™ APC anti-human Granulysin [Clone: DH2], Brilliant Violet 510™ anti-human Perforin [Clone: dG9], CD178 Monoclonal Antibody (SB93A), FITC.

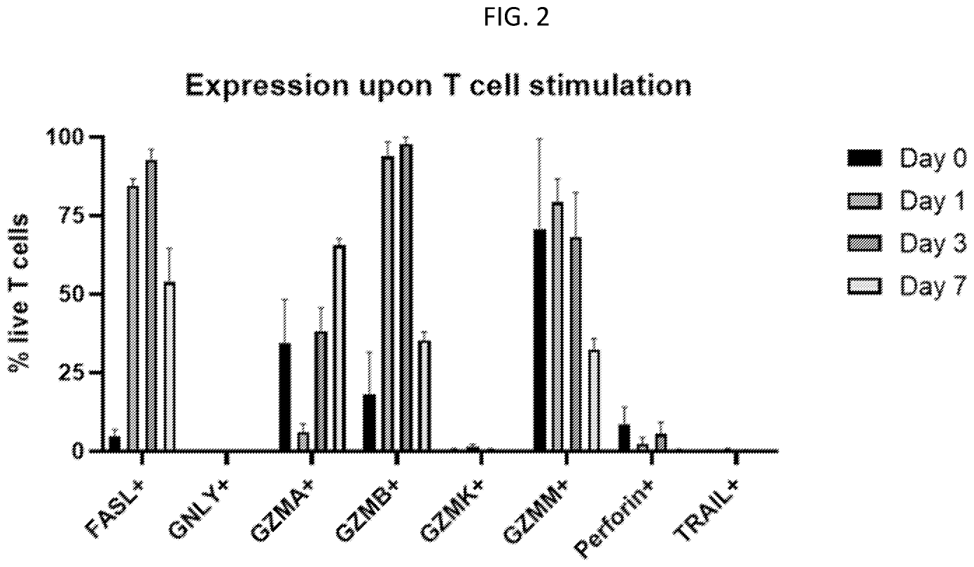

Based on the flow cytometry results (FIG. 2), granzymes A, B, and M were deemed as first priority for knockout because they are highly expressed and exert their cytotoxic effects through the pathway that we aim to utilize for therapeutic delivery of proteins. FasL was deemed as second priority for knockout, as it is also highly expression.

### Knockout of Individual Granzymes

Knockout of granzymes A, B, or M was performed in T cells from two donors by electroporation with 1.5 μg Cas9 mRNA and 1 μg of chemically modified sgRNA. Specifically, the sgRNAs were modified to include 2′-O-methyl 3′phosphorothioate (MS) on the 3′ and 5′ ends, which is known to increase longevity of the sgRNA in the cells for increased editing efficiency (Nat Biotechnol. (2015) 33 (9):985-989).

Multiple sgRNAs were designed to target each target gene and were tested for their knockout efficiency. The highest knockout efficiencies were achieved using the sgRNA sequences listed in Table 1 below. Following electroporation, cells were cultured in T cell expansion media with IL-2, IL-7, and IL-15 for 7 days and were then collected for genomic DNA extraction. To determine the knockout efficiency of the sgRNAs, the target loci were amplified using PCR and sequencing was performed. Synthego ICE analysis software was used to determine the most effective guides. The resulting ICE scores for each donor are listed below.

TABLE 1
sgRNA sequences were for their knockout efficiency in CD3+ T cells from two donors.
ICE and KO scores were calculated using the online Synthego ICE analysis platform.

| | | ICE score | ICE score | KO score | KO score |
|---|---|---|---|---|---|
| Target | sgRNA sequence | Donor 1 | Donor 2 | Donor 1 | Donor 2 |
| GZMA | ACCATGTAGGGTCTTG | 62 | 43 | 57 | 41 |
| | AATG (SEQ ID NO: 9) | | | | |
| GZMB | TTTCCTTCAGGGGAGA | 87 | 71 | 82 | 65 |
| | TCAT (SEQ ID NO: 10) | | | | |
| GZMM | CCCCAGCACCAGCCTC | 79 | 48 | 72 | 47 |
| | AGCT (SEQ ID NO: 11) | | | | |

### Knockout of Multiple Granzymes

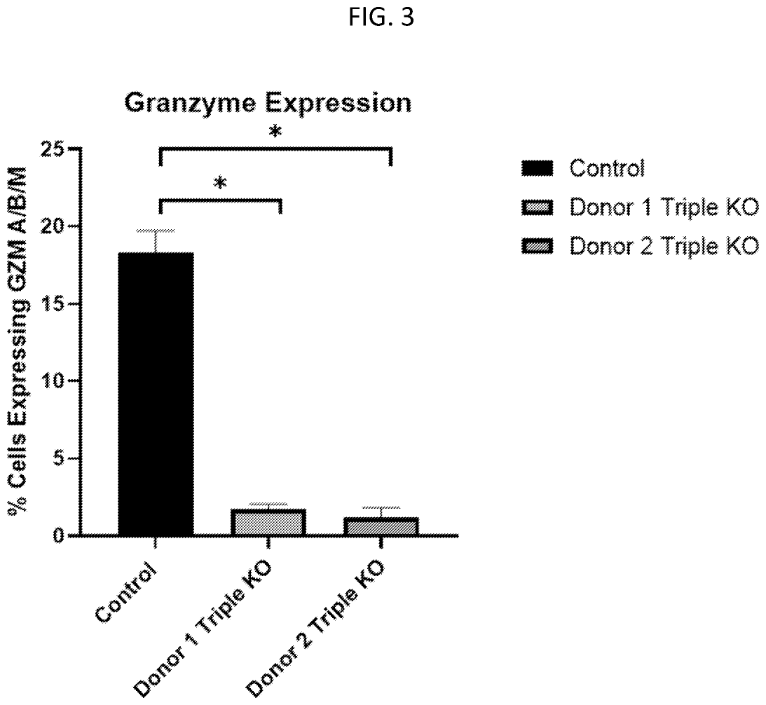

Concurrent knockout of granzymes A, B, and M in T cells from two donors was accomplished by electroporation of T cells with 1.5 μg Cas9 mRNA and 1 μg of each of the chemically modified sgRNAs listed in Table 1 above. Electroporated cells were cultured in expansion media for 5 days and then stained intracellularly for flow cytometry analysis of granzyme expression using the following fluorophore-conjugated antibodies: Pacific Blue™ anti-human Granzyme A [Clone: CB9], Alexa Fluor® 700 anti-human/mouse Granzyme B Recombinant [Clone: QA16A02], Granzyme M Mouse anti-Human, PE, Clone: 4B2G4, Invitrogen™. CD3 expression analysis was performed to isolate the population of single, live T cells. The results of this analysis (FIG. 3) revealed that roughly 18% of wild-type control cells expressed one or more of the granzymes targeted for knockout, whereas expression of one or more granzymes was reduced to less than 3% of the total single, live CD3+ population in the gene edited T cells.

### Introduction of CAR and GFP Reporter Constructs

We next used GZMA/GZMB/GZMM triple knockout T cells to generate anti-CD19 CAR T cells lacking expression of these three cytotoxic proteins. The anti-CD19 CAR was introduced into the triple knockout cells via transduction with a lentivirus encoding a CD19-targeting CAR. Specifically, we utilized a second-generation CAR, comprising a CD28 transmembrane domain 41BB and CD3ζ intracellular domains, that encodes an anti-CD19 scFv. An RQR8 sequence tag was included in the CAR to allow for quantification of CAR expression. To introduce the CAR, T cells were electroporated and transfected with this lentivirus (20 MOI) after 24 hours. In some cases, the construct encoding the CAR was co-delivered with a positive control construct comprising the MND promoter driving expression of GFP (5 MOI) or a construct comprising the U6 promoter driving expression of a fusion protein comprising a catalytically inactive form of granzyme B (dGZMB) linked to GFP via a glycine-serine linker (GSL) to test the ability of dGZMB to chaperone the lentiviral vector across the immunological synapse.

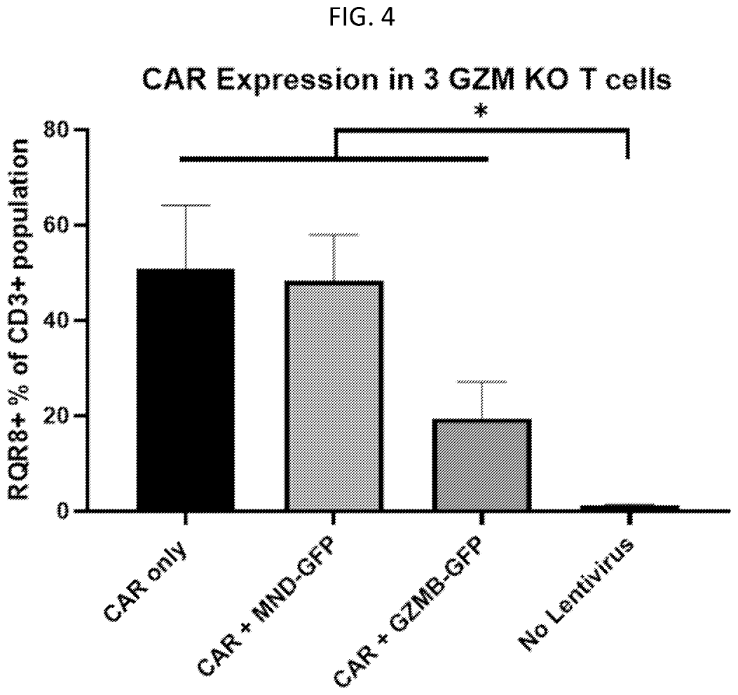

Flow cytometry was used to detect the percentage of CD3+ T cells expressing the RQR8 sequence tag as an indication of CAR expression. The results of this analysis (FIG. 4) revealed that CAR were able to be expressed in the genetically modified T cells that also expressed the fusion protein. Nevertheless, these results demonstrate that we are able to engineer functional T cells using either electroporation (which was used to knockout the granzyme proteins) or lentivirus (which was used to introduce the constructs encoding the CAR and fusion proteins).

### Killing Assay

To determine if knockout of granzymes A/B/M sufficiently reduces killing of target cells, we performed a co-culture experiment with CD19 expressing Raji cells. Raji cells were stained with Cytolight Rapid Orange fluorescent dye and plated at 6,000 cells per well in a poly-L-ornithine coated 96 well plate. Wild type or GZM A/B/M KO CD19 CAR T cells expressing MND driven dGZMB-GSL-GFP were used for this experiment. T cells were overlaid at a 1:1 effector to target ratio on the Raji cells after a 7 day rest period. Annexin V dye was added to the media in individual wells to label apoptotic cells. Live-cell phase-contrast and fluorescent images were taken at 20x magnification at intervals of 30 minutes for 48 hours by the Incucyte SX5 image acquisition system. Images were analyzed using Incucyte software to quantify the total number of Raji cells expressing Annexin V across conditions. Results from this analysis (FIG. 5) identified significant main effects of group F_{(3, 8)}=26.83 (P=0.0002), time F_{(2.086, 16.69)}=165.9 (P<0.0001), and a group by time interaction F_{(282, 752)}=17.38, (P<0.0001) are observed. The results indicate that addition of a CD19 CAR increases killing of target cells. However, significant differences are observed at multiple timepoints between WT CD19 CAR T cells and GZM A/B/M KO CD19 CAR T cells expressing MND driven dGZMB-GSL-GFP, suggesting that a GZM A/B/M KO along with the MND driven dGZMB-GSL-GFP reduce killing of target cells.

### Granzyme B Facilitated Transfer of GFP to Target Cells

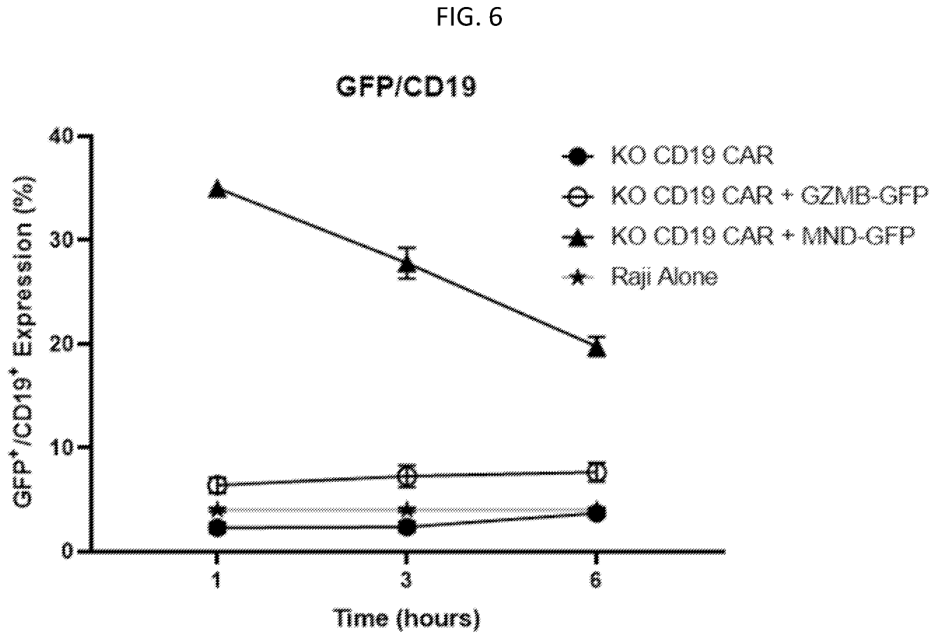

To determine if dGZMB can facilitate transfer of cargo proteins across the immunological synapse, we performed a similar co-culture experiment with Raji cells. Raji cells were labeled with CellTrace™ Violet before being placed in a 96-well plate for co-culture. A total of 100,000 GZMA/GZMB/GZMM triple knockout anti-CD19 CAR T, wild-type or expressing MND-GFP or MND-dGZMB-GFP, were plated per well at a 1:1 effector to target ratio. The engineered T cells were overlaid on Raji cells and incubated for 1, 3, and 6 hours in triplicate. At the end of the incubation, cells were placed on ice and were kept cold while they were stained for flow cytometry to reduce further interactions between the cell populations. Cells were stained to identify each cell as an effector cell (CD3+) or target cell (CD19+). Protein transfer was measured as the intracellular GFP signal within the Raji cells, which were isolated based on both CellTrace™ Violet staining and CD19 expression. Raji cells that were cultured alone or cultured with the triple knockout T cells that lacked a GFP construct served as negative controls. The results of this analysis (FIG. 6) revealed that Raji cells that were co-cultured with MND-GFP T cells exhibited up to 35% GFP positivity, whereas Raji cells that were co-cultured with GZMB-GFP exhibited nearly 10% GFP positivity. GFP positivity in targets was seen at the highest amounts in CAR-expressing T-cell cocultures, suggesting that immunological synapse formation between the CAR-T cells and target cells facilitated this transfer. Data is represented as mean±SD, n=3 per group per time point.

What is claimed:

1. A genetically modified lymphocyte comprising:
a) a mutation in one or more cytotoxic proteins that reduces or eliminates the expression of the one or more cytotoxic protein in the lymphocyte; and
b) a cargo molecule;
wherein the lymphocyte has reduced or no expression of the one or more cytotoxic protein.

2. The genetically modified lymphocyte of claim 1, wherein the cargo molecule comprises a protein, an RNA molecule, a DNA molecule or a small molecule.

3. The genetically modified lymphocyte of claim 1 or 2, wherein the lymphocyte comprises a first construct encoding the cargo molecule operably linked to a promoter.

4. The genetically modified lymphocyte of any one of claims 1-3, wherein the lymphocyte further comprises a chimeric antigen receptor (CAR).

5. The genetically modified lymphocyte of claim 4, wherein the CAR is an anti-CD19 CAR.

6. The genetically modified lymphocyte of any one of the preceding claims, wherein the one or more cytotoxic protein is selected from the group consisting of granzyme A (GZMA), granzyme B (GZMB), granzyme K (GZMK), and granzyme M (GZMM).

7. The genetically modified lymphocyte of any one of the preceding claims, wherein the lymphocyte comprises mutations in two or more cytotoxic proteins that reduce or eliminate the expression of the two or more cytotoxic proteins in the lymphocyte.

8. The genetically modified lymphocyte of claim 7, wherein the two or more cytotoxic proteins comprises GZMA, GZMB, and GZMM.

9. The genetically modified lymphocyte of any one of the preceding claims, wherein the cargo molecule is expressed as a fusion protein that comprises catalytically inactive granzyme, optionally wherein the catalytically inactive granzyme is catalytically inactive granzyme B (dGZMB).

10. The genetically modified lymphocyte of claim 9, wherein the cargo molecule is linked to dGZMB via a flexible glycine-serine linker.

11. The genetically modified lymphocyte of any one of the preceding claims, wherein the 10 cargo molecule is an RNA.

12. The genetically modified lymphocyte of claim 11, wherein the lymphocyte further comprising a second construct encoding a chaperone protein that binds to the RNA operably linked to a promoter.

13. The genetically modified lymphocyte of claim 11 or 12, wherein the RNA encoded by the first construct comprises MS2 stem loops, and wherein the chaperone protein encoded by the second construct comprises dGZMB and an MS2 binding protein.

14. The genetically modified lymphocyte of claim 13, wherein the second construct comprises SEQ ID NO:1 (dGZMB-GSL-MS2CP(N55K)).

15. The genetically modified lymphocyte of claim 13 or 14, wherein the RNA comprises a series of 6-24 MS2 stem loops.

16. The genetically modified lymphocyte of any one of the preceding claims, wherein the cargo molecule is a Cas9 protein, an RNA encoding Cas9, a guide nucleic acid (gNA) or combinations thereof.

17. The genetically modified lymphocyte of claim 16, wherein the first construct comprises SEQ ID NO:3 (dGZMB-GSL-Cas9), SEQ ID NO:7 (Cas9-MS2SLx24), or SEQ ID NO:8 (B2MsgRNA-MS2SL).

18. The genetically modified lymphocyte of any one of the preceding claims, wherein the promoter is MND or U6.

19. The genetically modified lymphocyte of any one of the preceding claims, wherein the lymphocyte is a T cell or a natural killer cell.

20. The genetically modified lymphocyte of any one of the preceding claims, wherein the lymphocyte exhibits reduced cytotoxicity as compared to an unmodified lymphocyte.

21. The genetically modified lymphocyte of any one of the preceding claims, wherein the cargo molecule is encapsulated in cytotoxic granules within the lymphocyte.

22. The genetically modified lymphocyte of any of the preceding claims, wherein the lymphocyte was differentiated from pluripotent stem cells.

23. A method for delivering a cargo molecule to a target cell, the method comprising co-culturing the target cell with the genetically modified lymphocyte of any one of the preceding claims, wherein the cargo molecule is delivered to the target cell.

24. The method of claim 23, wherein the lymphocyte comprises a CAR comprising a ligand-binding domain that specifically binds to an antigen present on the surface of the target cell.

25. The method of claim 24, wherein the antigen is CD19 and the target cell is a cancer cell.

26. The method of any one of claims 23-25, wherein the cargo molecule is secreted by the lymphocyte within cytotoxic granules.

27. A method for genetically modifying a target cell, the method comprising:
a) contacting the target cell with a genetically modified lymphocyte of any one of claims 1-22, wherein the cargo molecule in the genetically modified lymphocyte is a Cas9 protein, an RNA encoding Cas9, a guide nucleic acid (gNA), or a combination thereof.

28. The method of claim 27, wherein the genetically modified lymphocyte encodes a Cas9 protein and at least one guide RNA, and wherein activation of the lymphocyte transfers the Cas9 nuclease protein and at least one guide RNA to the target cell.

29. The method of claim 25 or 28 further comprising;
b) incubating the cells for a sufficient time for the target cell to be genetically modified by the cargo molecule.

30. The method of any one of claims 25-29, where the lymphocyte expresses a targeting protein that targets it to the target cell, preferably wherein the targeting protein is a chimeric antigen receptor.

31. The method of any one of claims 25-30, wherein the lymphocyte is a T cell.

32. A method for delivering one or more gene editing reagent to a target cell, the method comprising:
(a) providing a genetically modified lymphocyte comprising one or more exogenous nucleic acids encoding:
(i) a cargo molecule comprising a fusion protein comprising a catalytically dead granzyme (dGZ) fused to gene editing reagent, preferably a Cas9 protein, and one or more gNAs having complementarity to a target nucleic acid sequence to be genetically modified in the target cell; or
(ii) a cargo molecule comprising a fusion protein comprising a dGZ and an MS2 RNA-binding protein, and an RNA comprising one or more MS2 stem loops, and/or
(iii) a targeting molecule, preferably a chimeric antigen receptor (CAR), wherein the targeting molecule comprises a ligand-binding domain having specificity for an antigen present on the target cell,
wherein the genetically modified human lymphocyte expresses at last one cargo molecule, a targeting molecule, or both; and
(b) incubating the genetically modified lymphocyte with the target cell under conditions that promote activation of the genetically modified lymphocyte and delivery of the one or more cargo molecules to the target cell, preferably through perforin-induced membrane pores.

33. The method of claim 32, wherein the lymphocyte is a natural killer (NK) cell, cytotoxic T cell, regulatory T cell, or gamma-delta T cell.

34. The method of claim 33, wherein the cargo is encoded in a polynucleotide construct.

35. The method of any one of claims 32-34, wherein the lymphocyte is further modified:
a) to reduce or prevent expression of endogenous human granzyme A, granzyme H, granzyme M, tryptase-2, or combinations thereof;
b) to reduce or prevent expression of endogenous death receptor ligands TRAIL and FasL;
c) to reduce or prevent secretion of inflammatory cytokines IFNγ and TNFα; or
d) any combination of (a)-(c).

36. The method of any one of claims 32-35, wherein the ligand-binding domain of the CAR is specific for CD34 and wherein the target cell is a CD34⁺ hematopoietic stem cell (HSC).

37. The method of any one of claims 32-36, wherein the target cell is a T cell, B cell, CD34+ hematopoietic stem cell (HSC), a tumor cell, hepatocyte, liver stellate cell, neuron, microglia, fibroblast, keratinocyte, epithelial cell, hair follicle stem cell, or muscle cell progenitor.

38. The method of any one of claims 23-37, wherein the method is performed in vivo.

39. A genetically modified lymphocyte capable of gene editing a target cell, the lymphocyte comprising one or more nucleic acids encoding a (i) fusion protein comprising a catalytically dead granzyme B (dGZMB), granulysin, or catalytically inactive granulysin fused to a gene editing reagent, preferably a Cas9 protein, or dGZMB fused to a MS2 RNA-binding protein, and (ii) one or more gNAs having complementarity to a target nucleic acid sequence to be genetically modified in the target cell, wherein the human lymphocyte is genetically modified to express a chimeric antigen receptor (CAR) comprising a single-chain variant fragment (scFv) specific for the target cell.

40. The genetically modified lymphocyte of claim 39, wherein the human lymphocyte is a natural killer (NK) cell, cytotoxic T cell, regulatory T cell, or gamma-delta T cell.

41. The genetically modified lymphocyte of claim 39 or 40, wherein the human lymphocyte is further modified:
a) to reduce or prevent expression of endogenous human granzyme A, granzyme H, granzyme M, tryptase-2, or combinations thereof;
b) to reduce or prevent expression of endogenous death receptor ligands TRAIL and FasL; and/or
c) to reduce or prevent secretion of inflammatory cytokines IFNγ and TNFα.

42. The genetically modified lymphocyte of any one of claim 1-22 or 39-41, in which one or more of TRAIL, FASLG, IFNG, TNFA, GZMA, GZMH, GZIVIM, and TPSB2 genes have been disrupted by genetic knockout, wherein the genetically modified lymphocyte exhibits reduced expression of endogenous human granzyme A, granzyme H, granzyme M, tryptase-2, TRAIL, FasL or combinations thereof, and optionally further exhibits reduced secretion of IFNγ, TNFα or both relative to unmodified control cells.

43. The genetically modified lymphocyte of any one of claims 39-42, wherein the ligand-binding domain of the CAR is specific for CD34 and wherein the target cell is a CD34⁺ hematopoietic stem cell (HSC).

44. The genetically modified lymphocyte of any one of claims 39-43 further comprising one or more nucleic acids encoding a (i) fusion protein comprising granulysin or a catalytically inactive granulysin fused to Cas9 nuclease domain or a MS2 RNA binding protein.

45. The genetically modified lymphocyte of any one of claims 39-44, wherein the human lymphocyte is a natural killer (NK) cell, cytotoxic T cell, regulatory T cell, or gamma-delta T cell.

46. The genetically modified lymphocyte of any one of claims 39-45, wherein the ligand-binding domain of the CAR is specific for CD34 and wherein the target cell is a CD34⁺ hematopoietic stem cell (HSC).
